(19) United States
(12) Patent Application Publication
(10) Pub. No.: US 2026/0069163 A1
(43) Pub. Date: Mar. 12, 2026

(54) METHOD AND SYSTEM FOR BIOMARKERS DETECTION USING AI ANALYSIS OF COUGH AND VOICE SOUNDS

(71) Applicants: Radu VESTEMEAN, Thornhill (CA); Doru ROTOVEI, Tampa, FL (US)

(72) Inventors: Radu VESTEMEAN, Thornhill (CA); Doru ROTOVEI, Tampa, FL (US)

(21) Appl. No.: 19/394,194

(22) Filed: Nov. 19, 2025

Related U.S. Application Data
(60) Provisional application No. 63/723,632, filed on Nov. 22, 2024.

(51) Int. Cl. A61B 5/08; A61B 5/00; A61B 7/00; G10L 25/18; G10L 25/30; G10L 25/66; G16H 50/20

(52) U.S. Cl. CPC A61B 5/0823; A61B 5/0022; A61B 5/4803; A61B 5/7264; A61B 5/7275; A61B 5/7282; A61B 5/742; A61B 7/003; G10L 25/18; G10L 25/30; G10L 25/66; G16H 50/20

(57) ABSTRACT

A method and system for biomarker detection of cough and voice sounds using artificial analysis (AI) with a Convolutional Neural Network (CNN). Audio recordings of coughs and voice sounds are collected from a patient either in-person or remotely via telemedicine or telehealth platforms. The collected audio signals are transformed into waveform graphs and spectrograms for distinct processing pathways. AI CNN methods are used to classified cough types and detect respiratory diseases based on non-invasive acoustic signals. A final diagnostic report is prepared including one or more cough types and one or more disease categories and the calibrated probabilities for the patient. By analyzing the unique sound characteristics of coughs underlying respiratory conditions are accurately identified in real-time, offering a cost-effective and scalable diagnostic tool.

### CROSS REFERENCES TO RELATED APPICATIONS

The U.S. utility patent application claims priority from U.S. Provisional patent application No. 63/723,632, filed, Nov. 22, 2024, the contents of which are incorporated herein by reference.

### FIELD OF INVENTION

This invention relates to detecting biomarkers. More specifically, it relates to a method and system for biomarker detection of cough and voice sounds using artificial analysis (AI) of cough and voice sounds using Convolutional Neural Networks (CNN).

### BACKGROUND OF THE INVENTION

Respiratory diseases are among the leading causes of morbidity and mortality worldwide, with the United States seeing millions of cases annually. Conditions such as asthma, chronic obstructive pulmonary disease (COPD), pneumonia, and bronchitis account for a significant burden on the healthcare system. Early and accurate diagnosis of respiratory conditions is critical to providing timely treatment, improving patient outcomes, and reducing the economic burden on healthcare systems.

Asthma affects 26.8 million Americans, including 4.5 million children, and accounts for millions of emergency department visits and tens of billions of dollars in healthcare costs each year. While there is no cure, asthma can be managed and treated, helping those with the disease lead a healthier life.

About 11.7 million people, or 4.6% of adults, have been diagnosed with COPD (chronic obstructive pulmonary disease, chronic bronchitis, or emphysema). COPD is an obstructive lung disease that over time makes it harder to breathe. However, this number may be much higher because more than 18 million adults have evidence of impaired lung function, which may be undiagnosed COPD.

In the Region of the Americas in 2019, chronic respiratory diseases account for: 534,242 deaths in both sexes, 267,516 (50%) deaths in men and 266,725 (50%) in women. 35.8 deaths per 100,000 population (age-standardized), which was higher in men (42.2 deaths per 100,000) than in women (31.0 deaths per 100,000 population).

There are several problems associated with using traditional diagnostic methods for determining respiratory diseases.

One problem is that traditional diagnostic methods for respiratory diseases often require extensive clinical expertise, access to advanced equipment, and invasive procedures. These challenges are compounded in underserved or rural areas where access to healthcare facilities is limited.

Another problem is that a cough is a symptom of many different types of medical conditions. For example, a cough is a symptom for: (1) Allergies: Allergies to the nose or sinuses can cause a cough; (2) Asthma: Asthma can cause a cough, along with other symptoms like wheezing, chest tightness, and shortness of breath; (3) Gastroesophageal reflux disease (GERD): Stomach acid can irritate the throat or be breathed in, causing a cough; (4) Lung infections: Pneumonia, acute bronchitis, and other lung infections can cause a cough; (5) Sinusitis: Sinusitis with postnasal drip can cause a cough; (6) Medications: Some medications, like angiotensin-converting enzyme inhibitors (ACE inhibitors), can cause a cough; (7) COPD: chronic obstructive pulmonary disease, chronic bronchitis, or emphysema; (8) Smoking: Smoking can cause a cough, and a smoker's cough can be a symptom of COPD; (9) COVID-19: A chronic cough can be a symptom of COVID-19, and can linger for weeks or months after infection.

Another problem is that when a person is sick and has a cough it makes it difficult to seek medial treatment in a medical facility.

Another problem is that when a person is sick and has a cough, it is difficult during a telemedicine session to make a medical diagnosis based on a cough.

Another problem is that, misdiagnosis or delayed diagnosis of a cough can lead to prolonged suffering, increased healthcare costs, and potentially life-threatening complications for a patient.

Thus, it is desirable to solve some of the problems associated with dynamic diagnosis methods for respiratory diseases.

### SUMMARY OF THE INVENTION

In accordance with preferred embodiments of the present invention, some of the problems associated with dynamic diagnosis methods for respiratory diseases are overcome. A method and system for biomarker detection of cough and voice sounds using artificial analysis (AI) with Convolutional Neural Networks (CNN), is presented.

Audio recordings of coughs and voice sounds are collected from a patient either in-person or remotely via telemedicine and/or telehealth platforms. The collected audio signals are transformed into waveform graphs and spectrograms for distinct processing pathways. AI CNN methods are used to classified cough types and detect respiratory diseases based on non-invasive acoustic signals. A final diagnostic report is prepared including one or more cough types and one or more disease categories and the calibrated probabilities for the patient. By analyzing the unique sound characteristics of coughs underlying respiratory conditions are accurately identified in real-time, offering a cost-effective and scalable diagnostic tool.

The foregoing and other features and advantages of preferred embodiments of the present invention will be more readily apparent from the following detailed description. The detailed description proceeds with references to the accompanying drawings.

### BRIEF DESCRIPTION OF THE DRAWINGS

Preferred embodiments of the present invention are described with reference to the following drawings, wherein:

FIG. 1 is a block diagram illustrating an exemplary dynamic diagnosis of respiratory diseases system;

FIG. 2 is a block diagram illustrating an exemplary dynamic diagnosis of respiratory diseases display system;

FIG. 3 is a block diagram illustrating an exemplary networking protocol stack;

FIG. 4 is a block diagram illustrating an exemplary cloud communications network;

FIG. 5 is a block diagram illustrating an exemplary cloud storage object;

FIG. 6 is a block diagram illustrating wearable network devices;

FIG. 7 is a block diagram illustrating an exemplary dynamic diagnosis of respiratory diseases system;

FIG. 8 is a block diagram illustrating outputs from an exemplary dynamic diagnosis of respiratory diseases system;

FIGS. 9A and 9B are a flow diagram illustrating a method for dynamic diagnosis i of respiratory diseases;

FIG. 10 is a block diagram illustrating an exemplary data flow for the exemplary dynamic diagnosis of respiratory diseases system; and

FIG. 11 is a block diagram illustrating an exemplary data flow for the exemplary dynamic diagnosis of respiratory diseases system.

FIG. 12 is a flow diagram illustrating a method for dynamic diagnosis of respiratory diseases;

FIG. 13 is a flow diagram illustrating a method for dynamic diagnosis of respiratory diseases; and

FIG. 14 is a flow diagram illustrating a method for dynamic diagnosis of respiratory diseases.

### DETAILED DESCRIPTION OF THE INVENTION

### Exemplary Dynamic Diagnosis of Respiratory Diseases System

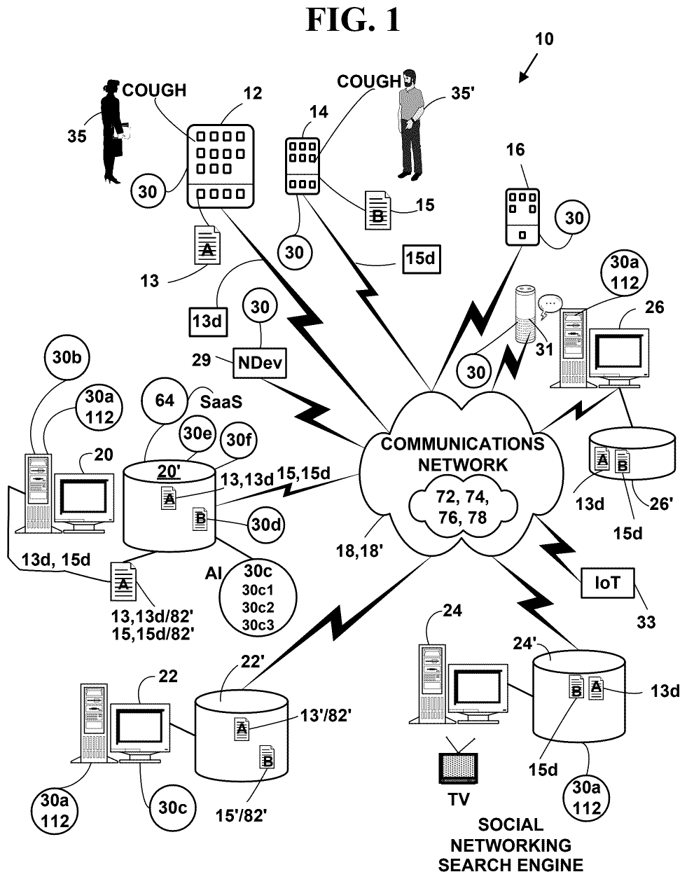

FIG. 1 is a block diagram illustrating an exemplary dynamic diagnosis of respiratory diseases system 10.

The exemplary system 10 includes, but is not limited to, one or more network devices, each with one or more processors and each with a non-transitory computer readable medium, one or more target network devices 12, 14, 16, etc. each with one or more processors and each with a non-transitory computer readable medium.

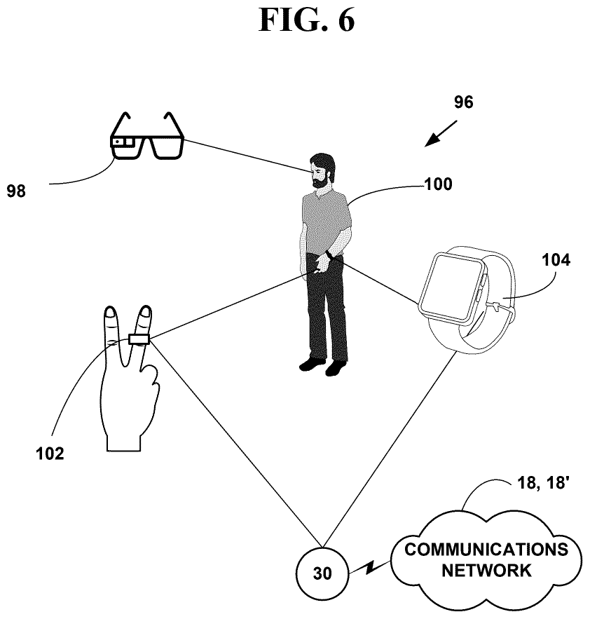

The one or more target network devices 12, 14, 16 (illustrated in FIG. 1 only as a tablet and two smart phones for simplicity) include, but are not limited to, desktop and laptop computers, tablet computers, mobile phones, non-mobile phones with displays, smart phones, Internet phones, Internet appliances, personal digital/data assistants (PDA), portable, handheld and desktop video game devices, cable television (CATV), satellite television (SATV) and Internet television set-top boxes, digital televisions including high definition television (HDTV), three-dimensional (3DTV) televisions, collectively NDev 29, smart speakers 31, Internet of Things (IoT) devices 33, wearable network devices 98-104 (FIG. 6) target network device interfaces on air (e.g., medical helicopter, etc.), water (e.g., water rescue, Coast Guard, etc.) or land (e.g., ambulance, fire truck, etc.) vehicles and/or other types of network devices.

A “smart phone” is a mobile phone 14 that offers more advanced computing ability and connectivity than a contemporary basic feature phone. Smart phones and feature phones may be thought of as handheld computers integrated with a mobile telephone, but while most feature phones are able to run applications based on platforms such as JAVA ME, a smart phone usually allows the user to install and run more advanced applications. Smart phones and/or tablet computers run complete operating system software providing a platform for application developers.

The tablet computers 12 include, but are not limited to, tablet computers such as the IPAD, by APPLE, Inc., the HP Tablet, by HEWLETT PACKARD, Inc., the PLAYBOOK, by RIM, Inc., the TABLET, by SONY, Inc., etc.

A “smart speaker” 31 is a type of wireless speaker and voice command device with an integrated virtual assistant that offers interactive actions and hands-free activation with the help of one “hot word” (or several “hot words”). Some smart speakers can also act as a smart device that utilizes Wi-Fi, BLUETOOTH®, other RFID, and other wireless protocol standards to extend usage beyond audio playback, such as to control home automation devices. This can include, but is not be limited to, features such as compatibility across a number of services and platforms, peer-to-peer connection through mesh networking, virtual assistants, and others. Each can have its own designated interface and features in-house, usually launched or controlled via application or home automation software. Some smart speakers also include a screen to show the user a visual response.

The IoT network devices 33, include but are not limited to, security cameras, doorbells with real-time video cameras, baby monitors, televisions, set-top boxes, lighting, heating (e.g., smart thermostats, etc.), ventilation, air conditioning (HVAC) systems, and appliances such as washers, dryers, robotic vacuums, air purifiers, ovens, refrigerators, freezers, toys, game platform controllers, game platform attachments (e.g., guns, googles, sports equipment, etc.), and/or other IoT network devices.

The target network devices 12, 14, 16, 29, 31, 33, 98-104 are in communications with a cloud communications network 18 or a non-cloud computing network 18′ via one or more wired and/or wireless communications interfaces. The cloud communications network 18, is also called a “cloud computing network” herein and the terms may be used interchangeably.

The plural target network devices 12, 14, 16, 29, 31, 33, 98-104 make requests 13, 15 for electronic messages (e.g., e-mail, SMS, MMS, RCS, DM, IM, social media, audio, video, network protocol (e.g., IP, etc., telephony, etc.) via the cloud communications network 18 or non-cloud communications network 18′.

The cloud communications network 18 and non-cloud communications network 18′ includes, but is not limited to, communications over a wire connected to the target network devices, wireless communications, and other types of communications using one or more communications and/or networking protocols.

Plural server network devices 20, 22, 24, 26 (only four of which are illustrated) each with one or more processors and a non-transitory computer readable medium include one or more associated databases 20′, 22′, 24′, 26′. The plural network devices 20, 22, 24, 26 are in communications with the one or more target devices 12, 14, 16, 29, 31, 33, 98-104 via the cloud communications network 18 and non-cloud communications network 18′.

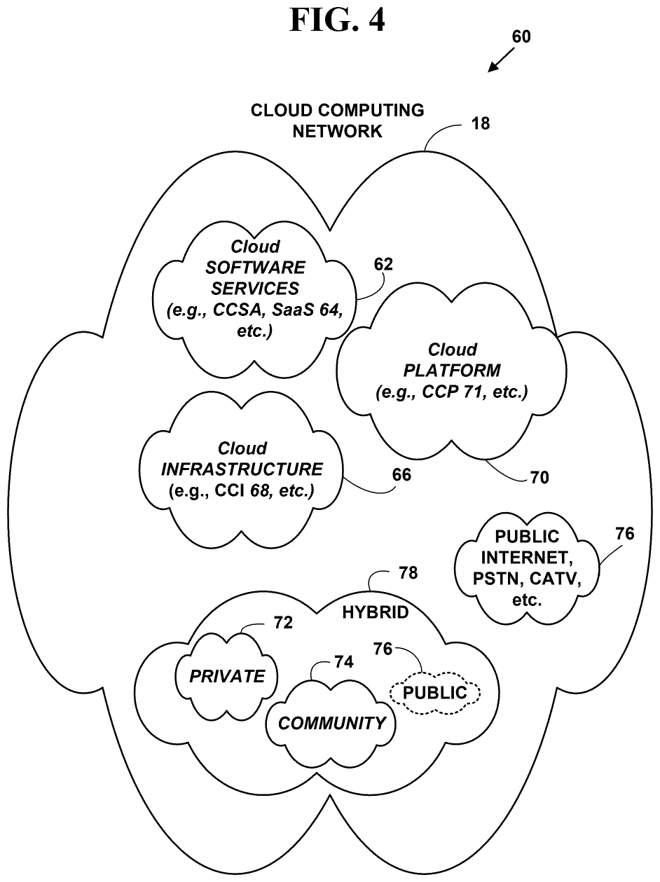

Plural server network devices 20, 22, 24, 26 (only four of which are illustrated) are physically located on one more public networks 76 (See FIG. 4), private networks 72, community networks 74 and/or hybrid networks 78 comprising the cloud network 18.

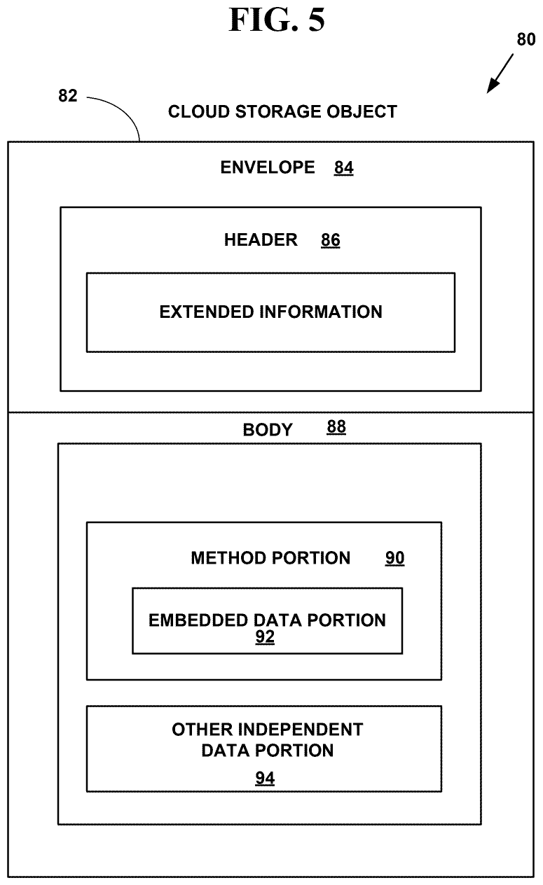

In one embodiment, one or more server network devices (e.g., 20, 22, 24, 26, etc.) store portions 13′, 15′ of the electronic content 13, 15 (e.g., audio data including cough data, e-mail, SMS, MMS, RCS, DM, IM, social media, network protocol, video, telephony, etc.) as cloud storage objects 13′/82, 15′/82 (FIG. 5) as is described herein.

The plural server network devices 20, 22, 24 26, may be connected to, but are not limited to, World Wide Web servers, Internet servers, search engine servers, vertical search engine servers, social networking site servers, file servers, other types of electronic information servers, and other types of server network devices (e.g., edge servers, firewalls, routers, gateways, etc.).

The plural server network devices 20, 22, 24, 26 also include, but are not limited to, network servers used for cloud computing providers, etc.

The cloud communications network 18 and non-cloud communications network 18′ includes, but is not limited to, a wired and/or wireless communications network comprising one or more portions of: the Internet, an intranet, a Local Area Network (LAN), a wireless LAN (WiLAN), a Wide Area Network (WAN), a Metropolitan Area Network (MAN), a Public Switched Telephone Network (PSTN), a Wireless Personal Area Network (WPAN) and other types of wired and/or wireless communications networks 18.

The cloud communications network 18 and non-cloud communications network 18′ includes one or more gateways, routers, bridges and/or switches. A gateway connects computer networks using different network protocols and/or operating at different transmission capacities. A router receives transmitted messages and forwards them to their correct destinations over the most efficient available route. A bridge is a device that connects networks using the same communications protocols so that information can be passed from one network device to another. A switch is a device that filters and forwards packets between network segments based on some pre-determined sequence (e.g., timing, sequence number, etc.).

An operating environment for the network devices of the exemplary electronic information display system 10 include a processing system with one or more high speed Central Processing Unit(s) (CPU), processors, one or more memories and/or other types of non-transitory computer readable mediums. In accordance with the practices of persons skilled in the art of computer programming, the present invention is described below with reference to acts and symbolic representations of operations or instructions that are performed by the processing system, unless indicated otherwise. Such acts and operations or instructions are referred to as being “computer-executed,” “CPU-executed,” or “processor-executed.”

It will be appreciated that acts and symbolically represented operations or instructions include the manipulation of electrical information by the CPU or processor. An electrical system represents data bits which cause a resulting transformation or reduction of the electrical information or biological information, and the maintenance of data bits at memory locations in a memory system to thereby reconfigure or otherwise alter the CPU's or processor's operation, as well as other processing of information. The memory locations where data bits are maintained are physical locations that have particular electrical, magnetic, optical, or organic properties corresponding to the data bits.

The data bits may also be maintained on a non-transitory computer readable medium including magnetic disks, optical disks, organic memory, and any other volatile (e.g., Random Access Memory (RAM)) or non-volatile (e.g., Read-Only Memory (ROM), flash memory, etc.) mass storage system readable by the CPU. The non-transitory computer readable medium includes cooperating or interconnected computer readable medium, which exist exclusively on the processing system or can be distributed among multiple interconnected processing systems that may be local or remote to the processing system.

### Exemplary Dynamic Diagnosis of Respiratory Diseases Display System

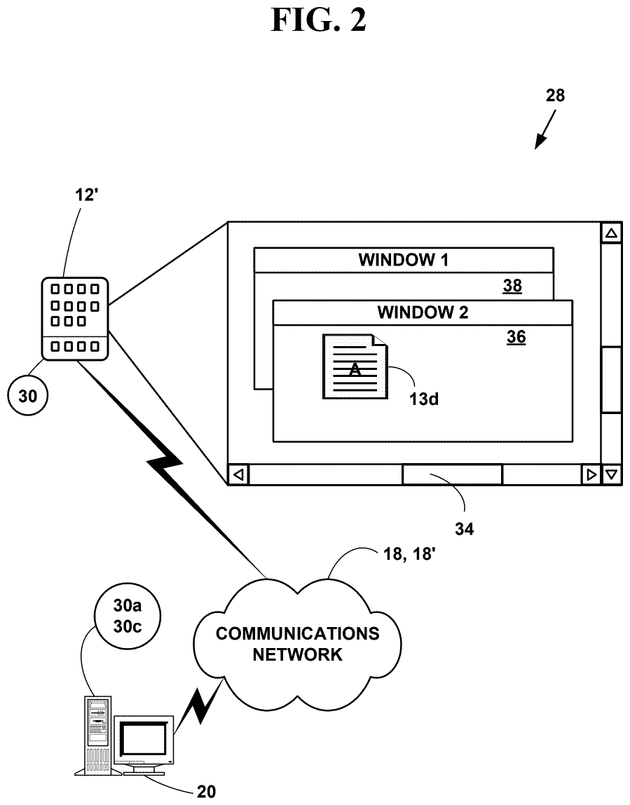

FIG. 2 is a block diagram illustrating an exemplary dynamic diagnosis of respiratory diseases display system 28.

The exemplary electronic message information display system 12′ includes, but is not limited to a target network device (e.g., 12, etc.) with a respiratory disease diagnosis application 30 and a display component 32. The application 30 presents a graphical user interface (GUI) 34 on the display 32 component. The GUI 32 presents a multi-window 36, 38, etc. (only two of which are illustrated) interface to a user. A server respiratory disease diagnosis application 30a is included on the server network device 20.

In one embodiment of the invention, the application 30 is a software application. However, the present invention is not limited to this embodiment and the application 30 can be hardware, firmware, hardware and/or any combination thereof. In one embodiment, the application 30 includes a mobile application for a smart phone, electronic tablet and/or other network device. In one embodiment, the application 30 includes web-browser based application. In one embodiment, the application 30 includes a web-chat client application. In another embodiment, the application 30a, 30b, 30c, 30d, 30e, 30f includes a cloud application used on a cloud communications network 18. However, the present invention is not limited these embodiments and other embodiments can be used to practice the invention

In another embodiment, a portion of the application 30 is executing on the target network devices 12, 14, 16, 29, 31, 33, 98-104 and another portion of the application 30a, 30b, 30c, 30d, 30e, 30f is executing on the server network devices 20, 22, 24, 26. The applications also include one or more library applications. However, the present invention is not limited these embodiments and other embodiments can be used to practice the invention.

### Exemplary Networking Protocol Stack

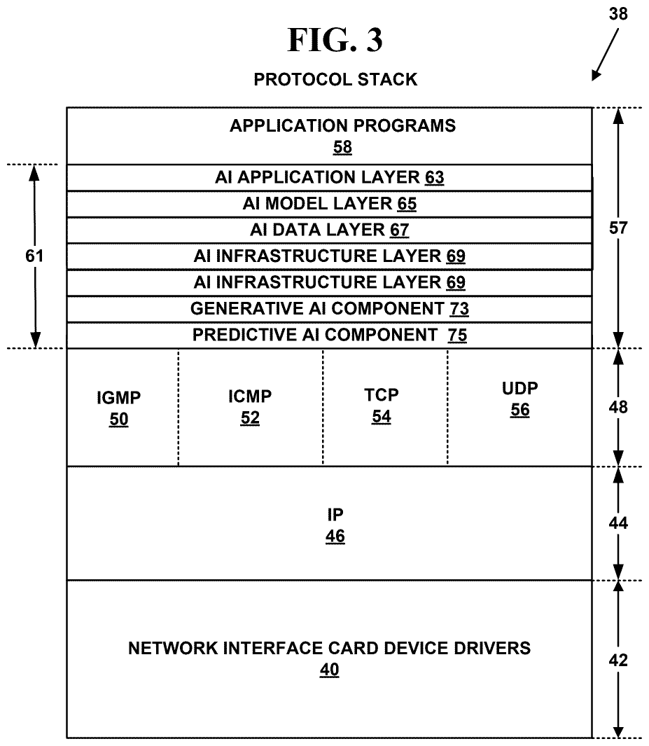

FIG. 3 a block diagram illustrating a layered protocol stack 38 for network devices in the dynamic diagnosis of respiratory diseases processing and display system 10.

The layered protocol stack 38 is described with respect to Internet Protocol (IP) suites comprising in general from lowest-to-highest, a link 42, network 44, transport 48 and application 56 layers. However, more or fewer layers could also be used, and different layer designations could also be used for the layers in the protocol stack 38 (e.g., layering based on the Open Systems Interconnection (OSI) model including from lowest-to-highest, a physical, data-link, network, transport, session, presentation and application layer.).

The network devices 12, 14, 16, 20, 22, 24, 26, 29, 31, 33, 98-104 are connected to the communication network 18 with Network Interface Card (NIC) cards including device drivers 40 in a link layer 42 for the actual hardware connecting the network devices 12, 14, 16, 20, 22, 24, 26, 39, 31, 33, 98-104 to the cloud communications network 18. For example, the NIC device drivers 40 may include a serial port device driver, a digital subscriber line (DSL) device driver, an Ethernet device driver, a wireless device driver, a wired device driver, etc. The device driver interface with the actual hardware being used to connect the network devices to the cloud communications network 18. The NIC cards have a medium access control (MAC) address that is unique to each NIC and unique across the whole cloud network 18. The Medium Access Control (MAC) protocol is used to provide a data link layer of an Ethernet LAN system and for other network systems.

Above the link layer 42 is a network layer 44 (also called the Internet Layer for Internet Protocol (IP) suites). The network layer 44 includes, but is not limited to, an IP layer 46.

IP 46 is an addressing protocol designed to route traffic within a network or between networks. However, more, fewer or other protocols can also be used in the network layer 44, and the present invention is not limited to IP 46. For more information on IP 46 see IETF RFC-791, incorporated herein by reference.

Above network layer 44 is a transport layer 48. The transport layer 48 includes, but is not limited to, an optional Internet Group Management Protocol (IGMP) layer 50, a Internet Control Message Protocol (ICMP) layer 52, a Transmission Control Protocol (TCP) layer 52 and a User Datagram Protocol (UDP) layer 54. However, more, fewer or other protocols could also be used in the transport layer 48.

Optional IGMP layer 50, hereinafter IGMP 50, is responsible for multicasting. For more information on IGMP 50 see RFC-1112, incorporated herein by reference. ICMP layer 52, hereinafter ICMP 52 is used for IP 46 control. The main functions of ICMP 52 include error reporting, reachability testing (e.g., pinging, etc.), route-change notification, performance, subnet addressing and other maintenance. For more information on ICMP 52 see RFC-792, incorporated herein by reference. Both IGMP 50 and ICMP 52 are not required in the protocol stack 38. ICMP 52 can be used alone without optional IGMP layer 50.

TCP layer 54, hereinafter TCP 54, provides a connection-oriented, end-to-end reliable protocol designed to fit into a layered hierarchy of protocols which support multi-network applications. TCP 54 provides for reliable inter-process communication between pairs of processes in network devices attached to distinct but interconnected networks. For more information on TCP 54 see RFC-793, incorporated herein by reference.

UDP layer 56, hereinafter UDP 56, provides a connectionless mode of communications with datagrams in an interconnected set of computer networks. UDP 56 provides a transaction-oriented datagram protocol, where delivery and duplicate packet protection are not guaranteed. For more information on UDP 56 see RFC-768, incorporated herein by reference. Both TCP 54 and UDP 56 are not required in protocol stack 38. Either TCP 54 or UDP 56 can be used without the other.

Above transport layer 48 is an application layer 57 where application programs 58 (e.g., 30, 30a, 30b, 30c, 30d, etc.) to carry out desired functionality for a network device reside. For example, the application programs 58 for the client network devices 12, 14, 16, 29, 31, 33, 98-104 may include web-browsers or other application programs, application program 30, while application programs for the server network devices 20, 22, 24, 26 may include other application programs (e.g., 30a, 30b, 30c, 30d, etc.).

In one embodiment, application program 30 includes an dynamic diagnosis of respiratory diseases detection application 30 on a target network device 12, 14, 16, 29, 31, 33, 98-104 and a dynamic diagnosis of respiratory diseases application 30a on a server network device 20, 22, 24, 26, including but not limited to, dynamic diagnosis of respiratory diseases application 30a and 30b, an Artificial Intelligence (AI) application 30c and/or other applications 30d, 30d, 30e, 30f. In one embodiment, the server event detection application 30a includes SaaS 64. However, the present invention is not limited to such an embodiment and more, fewer and/or other applications can be used to practice the invention.

However, the protocol stack 38 is not limited to the protocol layers illustrated and more, fewer or other layers and protocols can also be used in protocol stack 38. In addition, other protocols from the Internet Protocol suites (e.g., Simple Mail Transfer Protocol, (SMTP), Hyper Text Transfer Protocol (HTTP), File Transfer Protocol (FTP), Dynamic Host Configuration Protocol (DHCP), DNS, etc.), Short Message Peer-to-Peer (SMPP), and/or other protocols from other protocol suites may also be used in protocol stack 38.

In addition, markup languages such as HyperText Markup Language (HTML), Extensible Markup Language (XML) and others are used.

HyperText Markup Language (HTML) is a markup language for creating web pages and other information that can be displayed in a web browser.

HTML is written in the form of HTML elements consisting of tags enclosed in angle brackets within the web page content. HTML tags most commonly come in pairs although some tags represent empty elements and so are unpaired. The first tag in a pair is the start tag, and the second tag is the end tag (they are also called opening tags and closing tags). In between these tags web designers can add text, further tags, comments and other types of text-based content.

The purpose of a web browser is to read HTML documents and compose them into visible or audible web pages. The browser does not display the HTML tags, but uses the tags to interpret the content of the page.

HTML elements form the building blocks of all websites. HTML allows images and objects to be embedded and can be used to create interactive forms. It provides a means to create structured documents by denoting structural semantics for text such as headings, paragraphs, lists, links, quotes and other items. It can embed scripts written in languages such as JavaScript which affect the behavior of HTML web pages.

Extensible Markup Language (XML) is another markup language that defines a set of rules for encoding documents in a format that is both human-readable and machine-readable. It is defined in the XML 1.0 Specification produced by the W3C, the contents of which are incorporated by reference and several other related specifications, all free open standards.

XML a textual data format with strong support via Unicode for the languages of the world. Although the design of XML focuses on documents, it is widely used for the representation of arbitrary data structures, for example in web services. The oldest schema language for XML is the Document Type Definition (DTD). DTDs within XML documents define entities, which are arbitrary fragments of text and/or markup tags that the XML processor inserts in the DTD itself and in the XML document wherever they are referenced, like character escapes.

The Short Message Peer-to-Peer (SMPP) protocol in the telecommunications industry is an open, industry standard protocol designed to provide a flexible data communication interface for the transfer of short message data between External Short Messaging Entities, Routing Entities (ESME) and Short Message Service Center (SMSC).

Preferred embodiments of the present invention include network devices and wired and wireless interfaces that are compliant with all or part of standards proposed by the Institute of Electrical and Electronic Engineers (IEEE), International Telecommunications Union-Telecommunication Standardization Sector (ITU), European Telecommunications Standards Institute (ETSI), Internet Engineering Task Force (IETF), U.S. National Institute of Security Technology (NIST), American National Standard Institute (ANSI), Wireless Application Protocol (WAP) Forum, Bluetooth Forum, or the ADSL Forum.

### Wireless Interfaces

In one embodiment of the present invention, the wireless interfaces on network devices 12, 14, 16, 20, 22, 24, 26, 29, 31, 33, 98-104 include but are not limited to, IEEE 802.11a, 802.11b, 802.11g, 802.11n, 802.11ac, 802.11ax, 802.11be, 802.15.4 (ZigBee), “Wireless Fidelity” (Wi-Fi), “Worldwide Interoperability for Microwave Access” (WiMAX), ETSI High Performance Radio Metropolitan Area Network (HIPERMAN) or “RF Home” wireless interfaces. In another embodiment of the present invention, the wireless sensor device may include an integral or separate BLUETOOTH®, BLUETOOTH® Low Energy (BLE) and/or infra data association (IrDA), Machine-to-Machine (M2M), Near Field Communications (NFC), and/or other wireless infrared communications. However, the present invention is not limited to such embodiments, and other types of wireless interfaces can also be used to practice the invention.

802.11b is a short-range wireless network standard. The IEEE 802.11b standard defines wireless interfaces that provide up to 11 Mbps wireless data transmission to and from wireless devices over short ranges. 802.11a is an extension of the 802.11b and can deliver speeds up to 54 Mbps. 802.11g deliver speeds on par with 802.11a. However, other 802.11XX interfaces can also be used and the present invention is not limited to the 802.11 protocols defined. The IEEE 802.11a, 802.11b and 802.11g standards are incorporated herein by reference.

802.11ac is a Wi-Fi standard, also known as Wi-Fi 5, that provides significantly faster and more efficient wireless connections compared to its predecessor, 802.11n. It operates exclusively on the 5 GHz band and uses technologies like wider channels, more spatial streams, multiple input multiple output (MIMO), and improved data encoding to achieve multi-gigabit speeds, making it ideal for high-bandwidth activities like 4K streaming and online gaming.

802.11ax is the technical name for the Wi-Fi 6 standard, a next-generation wireless networking protocol designed to improve speed, efficiency, and capacity, especially in crowded network environments. It uses technologies like Orthogonal frequency-division multiple access (OFDMA) and enhanced multi-user multiple input multiple output (MU-MIMO) to handle more connected devices simultaneously with higher throughput and lower latency compared to its predecessor, 802.11ac (Wi-Fi 5).

802.11be is a formal designation for the Wi-Fi 7 wireless networking standard, which offers significantly higher speeds, lower latency, and improved efficiency compared to previous generations. Key advancements include wider channel widths (up to 320 MHz), enhanced modulation (4096-Quadrature Amplitude Modulation (QAM)), more spatial streams, and the ability to use multiple bands and links simultaneously to improve performance. This results in speeds up to 40 Gbps and makes it suitable for demanding applications like high-definition streaming, large file transfers, and real-time artificial realities and virtual realities.

Wi-Fi is a type of 802.11xx interface, whether 802.11b, 802.11a, 802.11ac, 802.11ax, 802.11be, dual-band, etc. Wi-Fi devices include an RF interface such as 2.4 GHz for 802.11b, 802.11g and others and 5 GHz for 802.11a, 802.11ac, 802.11ax, 802.11be and others.

802.15.4 (Zigbee) is low data rate network standard used for mesh network devices such as sensors, interactive toys, smart badges, remote controls, and home automation. The 802.15.4 standard provides data rates of 250 kbps, 40 kbps, and 20 kbps., two addressing modes; 16-bit short and 64-bit IEEE addressing, support for critical latency devices, such as joysticks, Carrier Sense Multiple Access/Collision Avoidance, (CSMA-CA) channel access, automatic network establishment by a coordinator, a full handshake protocol for transfer reliability, power management to ensure low power consumption for multi-month to multi-year battery usage and up to 16 channels in the 2.4 GHz Industrial, Scientific and Medical (ISM) band (Worldwide), 10 channels in the 915 MHz (US) and one channel in the 868 MHz band (Europe). The IEEE 802.15.4-2003 standard is incorporated herein by reference.

WiMAX is an industry trade organization formed by leading communications component and equipment companies to promote and certify compatibility and interoperability of broadband wireless access equipment that conforms to the IEEE 802.16XX and ETSI HIPERMAN. HIPERMAN is the European standard for metropolitan area networks (MAN).

The IEEE The 802.16a and 802.16g standards are wireless MAN technology standard that provides a wireless alternative to cable, DSL and T1/E1 for last mile broadband access. It is also used as complimentary technology to connect IEEE 802.11XX hot spots to the Internet.

The IEEE 802.16a standard for 2-11 GHz is a wireless MAN technology that provides broadband wireless connectivity to fixed, portable and nomadic devices. It provides up to 50-kilometers of service area range, allows users to get broadband connectivity without needing direct line of sight with the base station, and provides total data rates of up to 280 Mbps per base station, which is enough bandwidth to simultaneously support hundreds of businesses with T1/E1-type connectivity and thousands of homes with DSL-type connectivity with a single base station. The IEEE 802.16g provides up to 100 Mbps.

The IEEE 802.16e standard is an extension to the approved IEEE 802.16/16a/16g standard. The purpose of 802.16e is to add limited mobility to the current standard which is designed for fixed operation.

The ESTI HIPERMAN standard is an interoperable broadband fixed wireless access standard for systems operating at radio frequencies between 2 GHz and 11 GHz.

The IEEE 802.16a, 802.16e and 802.16g standards are incorporated herein by reference. WiMAX can be used to provide a WLP.

The ETSI HIPERMAN standards TR 101 031, TR 101 475, TR 101 493-1 through TR 101 493-3, TR 101 761-1 through TR 101 761-4, TR 101 762, TR 101 763-1 through TR 101 763-3 and TR 101 957 are incorporated herein by reference. ETSI HIPERMAN can be used to provide a WLP.

BLUETOOTH® includes a short-range wireless technology standard that is used for exchanging data between fixed and mobile devices over short distances. In the most widely used mode, transmission power is limited to 2.5 milliwatts, giving it a very short range of up to 10 meters. BLUETOOTH® Low Energy (BLE) includes a wireless technology that uses less power and has a longer battery life than previous BLUETOOTH® technologies. BLE is designed to operate on small batteries and in short-range, while still maintaining a similar communication range to other BLUETOOTH® technologies.

An “RFID tag” is an object that can be applied to or incorporated into a product, animal, or person for the purpose of identification and/or tracking using RF signals.

An “RFID sensor” is a device that measures a physical quantity and converts it into an RF signal which can be read by an observer or by an instrument (e.g., target network devices 12, 14, 16, 19, 20, 22, 24, 26, 29, 31, 98-104, server network devices 20, 22, 24, 26, etc.).

Near field communication (NFC)” is a set of standards for smartphones and similar network devices to establish radio communication with each other by touching them together or bringing them into close proximity, usually no more than a few centimeters. Present applications include contactless transactions, data exchange, and simplified setup of more complex communications such as Wi-Fi. Communication is also possible between an NFC device and an unpowered NFC chip, called a “tag” including radio frequency identifier (RFID) tags 99 and/or sensor.

NFC standards cover communications protocols and data exchange formats, and are based on existing radio-frequency identification (RFID) standards including ISO/IEC 14443 and FeliCa. These standards include ISO/IEC 1809 and those defined by the NFC Forum, all of which are incorporated by reference.

“Machine to machine (M2M)” refers to technologies that allow both wireless and wired systems to communicate with other devices of the same ability. M2M uses a device to capture an event (such as option purchase, etc.), which is relayed through a network (wireless, wired cloud, etc.) to an application (software program), that translates the captured event into meaningful information. Such communication was originally accomplished by having a remote network of machines relay information back to a central hub for analysis, which would then be rerouted into a system like a personal computer.

However, modern M2M communication has expanded beyond a one-to-one connection and changed into a system of networks that transmits data many-to-one and many-to-many to plural different types of devices and appliances. The expansion of IP networks across the world has made it far easier for M2M communication to take place and has lessened the amount of power and time necessary for information to be communicated between machines.

However, the present invention is not limited to such wireless interfaces and wireless networks and more, fewer and/or other wireless interfaces can be used to practice the invention.

In one embodiment, the plural server network devices 20, 22, 24, 26 include a connection to plural network interface cards (NICs) in a backplane connected to a communications bus. The NIC cards provide gigabit/second (1×10⁹ bits/second) communications speed of electronic information. This allows “scaling out” for fast electronic content retrieval. The NICs are connected to the plural server network devices 20, 22, 24, 26 and the cloud communications network 18. However, the present invention is not limited to the NICs described and other types of NICs in other configurations and connections with and/or without buses can also be used to practice the invention.

In one embodiment, of the invention, the wireless interfaces also include wireless personal area network (WPAN) interfaces. As is known in the art, a WPAN is a personal area network for interconnecting devices centered around an individual person's devices in which the connections are wireless. A WPAN interconnects all the ordinary computing and communicating devices that a person has on their desk (e.g. computer, etc.) or carry with them (e.g., PDA, mobile phone, smart phone, table computer two-way pager, etc.).

A key concept in WPAN technology is known as “plugging in.” In the ideal scenario, when any two WPAN-equipped devices come into close proximity (within several meters and/or feet of each other) or within a few miles and/or kilometers of a central server (not illustrated), they can communicate via wireless communications as if connected by a cable. WPAN devices can also lock out other devices selectively, preventing needless interference or unauthorized access to secure information. Zigbee is one wireless protocol used on WPAN networks such as cloud communications network 18 or non-cloud communications network 18′.

In one specific embodiment, the one or more target network devices 12, 14, 16, 20, 22, 24, 26, 29, 31, 33, 98-104 and one or more server network devices 20, 22, 24, 26 and the AEDAAD 19 communicate with each other and/or other network devices with near field communications (NFC) and/or machine-to-machine (M2M) communications. However, the present invention is not to such an embodiment and other embodiments can be used to practice the invention

### Wired Interfaces

In one embodiment of the present invention, the wired interfaces include wired interfaces and corresponding networking protocols for wired connections to the Public Switched Telephone Network (PSTN) and/or a cable television network (CATV) and/or satellite television networks (SATV) and/or three-dimensional television (3DTV), including HDTV that connect the network devices 12, 14, 16, 19, 20, 22, 24, 26, 29, 31, 33, 98-104 via one or more twisted pairs of copper wires, digital subscriber lines (e.g. DSL, ADSL, VDSL, etc.) coaxial cable, fiber optic cable, other connection media or other connection interfaces. The PSTN is any public switched telephone network provided by AT&T, GTE, Sprint, MCI, SBC, Verizon and others. The CATV is any cable television network provided by the Comcast, Time Warner, etc. However, the present invention is not limited to such wired interfaces and more, fewer and/or other wired interfaces can be used to practice the invention.

### Television Services

In one embodiment, the cloud applications 30, 30a, 30b, 30c, 30d, 30e, 30f provide cloud SaaS 64 services and/or non-cloud application services from television services over the cloud communications network 18 or application services over the non-cloud communications network 18′. The television services include digital television services, including, but not limited to, cable television, satellite television, high-definition television, three-dimensional, televisions and other types of network devices.

However, the present invention is not limited to such television services and more, fewer and/or other television services can be used to practice the invention.

### Internet Television Services

In one embodiment, the cloud applications 30, 30a, 30b, 30c, 30d, 30e, 30f provide cloud SaaS 64 services and/or non-cloud application services from Internet television services over the cloud communications network 18 or non-cloud communications network 18′. The television services include Internet television, Web-TV, and/or Internet Protocol Television (IPtv) and/or other broadcast television services.

“Internet television” allows users to choose a program or the television show they want to watch from an archive of programs or from a channel directory. The two forms of viewing Internet television are streaming content directly to a media player or simply downloading a program to a viewer's set-top box, game console, computer, or other network device.

“Web-TV” delivers digital content via broadband and mobile networks. The digital content is streamed to a viewer's set-top box, game console, computer, or other network device.

“Internet Protocol television (IPtv)” is a system through which Internet television services are delivered using the architecture and networking methods of the Internet Protocol Suite over a packet-switched network infrastructure, e.g., the Internet and broadband Internet access networks, instead of being delivered through traditional radio frequency broadcast, satellite signal, and cable television formats.

However, the present invention is not limited to such Internet Television services and more, fewer and/or other Internet Television services can be used to practice the invention.

### General Search Engine Services

In one embodiment, the cloud applications 30, 30a, 30b, 30c, 30d, 30e, 30f provide cloud SaaS 64 services and/or non-cloud application services from general search engine services. A search engine is designed to search for information on a cloud communications network 18 or non-cloud communications network 18′ such as the Internet including World Wide Web servers, HTTP, FTP servers etc. The search results are generally presented in a list of electronic results. The information may consist of web pages, images, electronic information, multimedia information, and other types of files. Some search engines also mine data available in databases or open directories. Unlike web directories, which are maintained by human editors, search engines typically operate algorithmically and/or are a mixture of algorithmic and human input.

In one embodiment, the cloud applications 30, 30a, 30b, 30c, 30d, 30e, 30f provide cloud SaaS 64 services and/or non-cloud application services from general search engine services. In another embodiment, the cloud applications 30, 30a, 30b, 30c, 30d, 30e, 30f provide general search engine services by interacting with one or more other public search engines (e.g., GOOGLE, BING, YAHOO, etc.) and/or private search engine services.

In another embodiment, the cloud applications 30, 30a, 30b, 30c, 30d, 30e, 30f provide cloud SaaS 64 services and/or non-cloud application services from specialized search engine services, such as vertical search engine services by interacting with one or more other public vertical search engines and/or private search engine services.

However, the present invention is not limited to such general and/or vertical search engine services and more, fewer and/or other general search engine services can be used to practice the invention.

### Social Networking Services

In one embodiment, the cloud applications 30, 30a, 30b, 30c, 30d, 30e, 30f provide cloud SaaS 64 services and/or non-cloud application services from one more social networking services including to/from one or more social networking web-sites (e.g., FACEBOOK, YOUTUBE, TWITTER, INSTAGRAM, etc.). The social networking web-sites also include, but are not limited to, social couponing sites, dating web-sites, blogs, RSS feeds, and other types of information web-sites in which messages can be left or posted for a variety of social activities.

However, the present invention is not limited to the social networking services described and other public and private social networking services can also be used to practice the invention.

### Security and Encryption

Network devices 12, 14, 16, 20, 22, 24, 26, 29. 31, 33, 98-104 with wired and/or wireless interfaces of the present invention include one or more of the security and encryptions techniques discussed herein for secure communications on the cloud communications network 18 or non-cloud communications network 18′.

Application programs 58 (FIG. 2) include security and/or encryption application programs integral to and/or separate from the applications 30, 30a, 30b, 30c, 30d. Security and/or encryption programs may also exist in hardware components on the network devices (12, 14, 16, 20, 22, 24, 26, 29, 31, 98-104) described herein and/or exist in a combination of hardware, software and/or firmware.

Wireless Encryption Protocol (WEP) (also called “Wired Equivalent Privacy) is a security protocol for WiLANs defined in the IEEE 802.11b standard. WEP is cryptographic privacy algorithm, based on the Rivest Cipher 4 (R194) encryption engine, used to provide confidentiality for 802.11b wireless data.

R194 is cipher designed by RSA Data Security, Inc. of Bedford, Massachusetts, which can accept encryption keys of arbitrary length, and is essentially a pseudo random number generator with an output of the generator being XORed with a data stream to produce encrypted data.

One problem with WEP is that it is used at the two lowest layers of the OSI model, the physical layer and the data link layer, therefore, it does not offer end-to-end security. One another problem with WEP is that its encryption keys are static rather than dynamic. To update WEP encryption keys, an individual has to manually update a WEP key. WEP also typically uses 40-bit static keys for encryption and thus provides “weak encryption,” making a WEP device a target of hackers.

The IEEE 802.11 Working Group is working on a security upgrade for the 802.11 standard called “802.11i.” This supplemental draft standard is intended to improve WiLAN security. It describes the encrypted transmission of data between systems 802.11X WiLANs. It also defines new encryption key protocols including the Temporal Key Integrity Protocol (TKIP). The IEEE 802.11i draft standard, version 4, completed Jun. 6, 2003, is incorporated herein by reference.

The 802.11i standard is based on 802.1x port-based authentication for user and device authentication. The 802.11i standard includes two main developments: Wi-Fi Protected Access (WPA) and Robust Security Network (RSN).

WPA uses the same R194 underlying encryption algorithm as WEP. However, WPA uses TKIP to improve security of keys used with WEP. WPA keys are derived and rotated more often than WEP keys and thus provide additional security. WPA also adds a message-integrity-check function to prevent packet forgeries.

RSN uses dynamic negotiation of authentication and selectable encryption algorithms between wireless access points and wireless devices. The authentication schemes proposed in the draft standard include Extensible Authentication Protocol (EAP). One proposed encryption algorithm is an Advanced Encryption Standard (AES) encryption algorithm.

Dynamic negotiation of authentication and encryption algorithms lets RSN evolve with the state of the art in security, adding algorithms to address new threats and continuing to provide the security necessary to protect information that WiLANs carry.

The NIST developed a new encryption standard, the Advanced Encryption Standard (AES) to keep government information secure. AES is intended to be a stronger, more efficient successor to Triple Data Encryption Standard (3DES).

DES is a popular symmetric-key encryption method developed in 1975 and standardized by ANSI in 1981 as ANSI X.3.92, the contents of which are incorporated herein by reference. As is known in the art, 3DES is the encrypt-decrypt-encrypt (EDE) mode of the DES cipher algorithm. 3DES is defined in the ANSI standard, ANSI X9.52-1998, the contents of which are incorporated herein by reference. DES modes of operation are used in conjunction with the NIST Federal Information Processing Standard (FIPS) for data encryption (FIPS 46-3, October 1999), the contents of which are incorporated herein by reference.

The NIST approved a FIPS for the AES, FIPS-197. This standard specified “Rijndael” encryption as a FIPS-approved symmetric encryption algorithm that may be used by U.S. Government organizations (and others) to protect sensitive information. The NIST FIPS-197 standard (AES FIPS PUB 197, November 2001) is incorporated herein by reference.

The NIST approved a FIPS for U.S. Federal Government requirements for information technology products for sensitive but unclassified (SBU) communications. The NIST FIPS Security Requirements for Cryptographic Modules (FIPS PUB 140-2, May 2001) is incorporated herein by reference.

RSA is a public key encryption system which can be used both for encrypting messages and making digital signatures. The letters RSA stand for the names of the inventors: Rivest, Shamir and Adleman. For more information on RSA, see U.S. Pat. No. 4,405,829, now expired and incorporated herein by reference.

“Hashing” is the transformation of a string of characters into a usually shorter fixed-length value or key that represents the original string. Hashing is used to index and retrieve items in a database because it is faster to find the item using the shorter hashed key than to find it using the original value. It is also used in many encryption algorithms.

Secure Hash Algorithm (SHA), is used for computing a secure condensed representation of a data message or a data file. When a message of any length <2⁶⁴ bits is input, the SHA-1 produces a 160-bit output called a “message digest.” The message digest can then be input to other security techniques such as encryption, a Digital Signature Algorithm (DSA) and others which generates or verifies a security mechanism for the message. SHA-512 outputs a 512-bit message digest. The Secure Hash Standard, FIPS PUB 180-1, Apr. 17, 1995, is incorporated herein by reference.

Message Digest-5 (MD-5) takes as input a message of arbitrary length and produces as output a 128-bit “message digest” of the input. The MD5 algorithm is intended for digital signature applications, where a large file must be “compressed” in a secure manner before being encrypted with a private (secret) key under a public-key cryptosystem such as RSA. The IETF RFC-1321, entitled “The MD5 Message-Digest Algorithm” is incorporated here by reference.

Providing a way to check the integrity of information transmitted over or stored in an unreliable medium such as a wireless network is a prime necessity in the world of open computing and communications. Mechanisms that provide such integrity check based on a secret key are called “message authentication codes” (MAC). Typically, message authentication codes are used between two parties that share a secret key in order to validate information transmitted between these parties.

Keyed Hashing for Message Authentication Codes (HMAC), is a mechanism for message authentication using cryptographic hash functions. HMAC is used with any iterative cryptographic hash function, e.g., MD5, SHA-1, SHA-512, etc. in combination with a secret shared key. The cryptographic strength of HMAC depends on the properties of the underlying hash function. The IETF RFC-2101, entitled “HMAC: Keyed-Hashing for Message Authentication” is incorporated here by reference.

An Electronic Code Book (ECB) is a mode of operation for a “block cipher,” with the characteristic that each possible block of plaintext has a defined corresponding cipher text value and vice versa. In other words, the same plaintext value will always result in the same cipher text value. Electronic Code Book is used when a volume of plaintext is separated into several blocks of data, each of which is then encrypted independently of other blocks. The Electronic Code Book has the ability to support a separate encryption key for each block type.

Diffie and Hellman (DH) describe several different group methods for two parties to agree upon a shared secret in such a way that the secret will be unavailable to eavesdroppers. This secret is then converted into various types of cryptographic keys. A large number of the variants of the DH method exist including ANSI X9.42. The IETF RFC-2631, entitled “Diffie-Hellman Key Agreement Method” is incorporated here by reference.

The HyperText Transport Protocol (HTTP) Secure (HTTPs), is a standard for encrypted communications on the World Wide Web. HTTPs is actually just HTTP over a Secure Sockets Layer (SSL). For more information on HTTP, see IETF RFC-2616 incorporated herein by reference.

The SSL protocol is a protocol layer which may be placed between a reliable connection-oriented network layer protocol (e.g. TCP/IP) and the application protocol layer (e.g. HTTP). SSL provides for secure communication between a source and destination by allowing mutual authentication, the use of digital signatures for integrity, and encryption for privacy.

The SSL protocol is designed to support a range of choices for specific security methods used for cryptography, message digests, and digital signatures. The security methods are negotiated between the source and destination at the start of establishing a protocol session. The SSL 2.0 protocol specification, by Kipp E. B. Hickman, 1995 is incorporated herein by reference. More information on SSL is available at the domain name.

Transport Layer Security (TLS) provides communications privacy over the Internet. The protocol allows client/server applications to communicate over a transport layer (e.g., TCP) in a way that is designed to prevent eavesdropping, tampering, or message forgery. For more information on TLS see IETF RFC-2246, incorporated herein by reference.

In one embodiment, the security functionality includes Cisco Compatible EXtensions (CCX). CCX includes security specifications for makers of 802.11xx wireless LAN chips for ensuring compliance with Cisco's proprietary wireless security LAN protocols. As is known in the art, Cisco Systems, Inc. of San Jose, California is supplier of networking hardware and software, including router and security products.

However, the present invention is not limited to such security and encryption methods described herein and more, fewer and/or other types of security and encryption methods can be used to practice the invention. The security and encryption methods described herein can also be used in various combinations and/or in different layers of the protocol stack 38 with each other.

### Cloud Computing Networks

FIG. 4 is a block diagram 60 illustrating an exemplary cloud computing network 18. The cloud computing network 18 is also referred to as a “cloud communications network” 18. However, the present invention is not limited to this cloud computing model and other cloud computing models can also be used to practice the invention. The exemplary cloud communications network includes both wired and/or wireless components of public and private networks.

In one embodiment, the cloud computing network 18 includes a cloud communications network 18 comprising plural different cloud component networks 72, 74, 76, 78. “Cloud computing” is a model for enabling, on-demand network access to a shared pool of configurable computing resources (e.g., public and private networks, servers, storage, applications, and services) that are shared, rapidly provisioned and released with minimal management effort or service provider interaction.

This exemplary cloud computing model for electronic information retrieval promotes availability for shared resources and comprises: (1) cloud computing essential characteristics; (2) cloud computing service models; and (3) cloud computing deployment models. However, the present invention is not limited to this cloud computing model and other cloud computing models can also be used to practice the invention.

Exemplary cloud computing essential characteristics appear in Table 1. However, the present invention is not limited to these essential characteristics and more, fewer or other characteristics can also be used to practice the invention.

TABLE 1

1. On-demand dynamic diagnosis of respiratory diseases interoperability
services. Automatic online dynamic diagnosis of respiratory diseases
services can unilaterally provision computing capabilities, such as
server time and network storage, as needed automatically without
requiring human interaction with each network server on the cloud
communications network 18.
2. Broadband network access. Automatic online dynamic diagnosis of
respiratory diseases services capabilities are available over plural
broadband communications networks and accessed through standard
mechanisms that promote use by heterogeneous thin or thick client
platforms (e.g., mobile phones, smart phones 14, tablet computers 12,
laptops, PDAs, etc.). The broadband network access includes high
speed network access such as 3G, 4G and 5G wireless and/or wired and
broadband and/or ultra-broad band (e.g., WiMAX, etc.) network access.
3. Resource pooling. Automatic online dynamic diagnosis of respiratory
diseases resources are pooled to serve multiple requesters using a multi-
tenant model, with different physical and virtual resources dynamically
assigned and reassigned according to demand. There is location
independence in that a requester of services has no control and/ or
knowledge over the exact location of the provided by the dynamic
diagnosis of respiratory diseases service resources but may be able to
specify location at a higher level of abstraction (e.g., country, state, or
data center). Examples of pooled resources include storage, processing,
memory, network bandwidth, virtual server network device and virtual
target network devices.
4. Rapid elasticity. Capabilities can be rapidly and elastically provisioned, in
some cases automatically, to quickly scale out and rapidly released to
quickly scale for education interoperability service collaboration. For
automatic dynamic diagnosis of respiratory diseases ervices, multi-
media collaboration converters, the automatic online dynamic diagnosis
of respiratory diseases processing services collaboration and analytic
conversion capabilities available for provisioning appear to be unlimited
and can be used in any quantity at any time.
5. Measured Services. Cloud computing systems automatically control and
optimize resource use by leveraging a metering capability at some level
of abstraction appropriate to the type of automatic online dynamic
diagnosis of respiratory diseases services (e.g., storage, processing,
bandwidth, custom electronic content retrieval applications, etc.).
Electronic automatic dynamic diagnosis of respiratory diseases
processing services collaboration conversion usage is monitored,
controlled, and reported providing transparency for both the automatic
online dynamic diagnosis of respiratory diseases services provider and
the dynamic diagnosis of respiratory diseases interoperability service
requester of the utilized electronic content storage retrieval service.

Exemplary cloud computing service models illustrated in FIG. 4 appear in Table 2. However, the present invention is not limited to these service models and more, fewer or other service models can also be used to practice the invention.

TABLE 2
1. Cloud Computing Software Applications 62 for dynamic diagnosis of
respiratory diseases services (CCSA, SaaS 64). The capability to use
the provider's applications 30, 30a, 30b, 30c, 30d, 30e, 30f running on a
cloud infrastructure 66. The cloud computing applications 62, are
accessible from the server network device 20 from various client
devices 12, 14, 16 through a thin client interface such as a web browser,
etc. The user does not manage or control the underlying cloud
infrastructure 66 including network, servers, operating systems, storage,
or even individual application 30, 30a, 30b, 30c, 30d, 30e, 30f
capabilities, with the possible exception of limited user-specific
application configuration settings.
2. Cloud Computing Infrastructure 66 for dynamic diagnosis of respiratory
diseases services (CCI 68). The capability provided to the user is to
provision processing, storage and retrieval, networks 18, 72, 74, 76, 78
and other fundamental computing resources where the consumer is able
to deploy and run arbitrary software, which can include operating
systems and applications 30, 30a, 30b, 30c, 30d. The user does not
manage or control the underlying cloud infrastructure 66 but has control
over operating systems, storage, deployed applications, and possibly
limited control of select networking components (e.g., host firewalls,
etc.).
3. Cloud Computing Platform 70 for dynamic diagnosis of respiratory diseases
services (CCP 71). The capability provided to the user to deploy onto
the cloud infrastructure 66 created or acquired applications created
using programming languages and tools supported servers 20, 22, 24,
26, etc. The user not manage or control the underlying cloud
infrastructure 66 including network, servers, operating systems, or
storage, but has control over the deployed applications 30a, 30b, 30c,
30d, 30e, 30f and possibly application hosting environment
configurations.

Exemplary cloud computing deployment models appear in Table 3. However, the present invention is not limited to these deployment models and more, fewer or other deployment models can also be used to practice the invention.

TABLE 3
1. Private cloud network 72. The cloud network infrastructure is operated
solely for dynamic diagnosis of respiratory diseases services. It may be
managed by the electronic content retrieval or a third party and may
exist on premise or off premise.
2. Community cloud network 74. The cloud network infrastructure is shared by
several different organizations and supports a specific electronic content
storage and retrieval community that has shared concerns (e.g., mission,
security requirements, policy, compliance considerations, etc.). It may
be managed by the different organizations or a third party and may exist
on premise or off premise.
3. Public cloud network 76. The cloud network infrastructure such as the
Internet, PSTN, SATV, CATV, Internet TV, etc. is made available to
the general public or a large industry group and is owned by one or
more organizations selling cloud services.
4. Hybrid cloud network 78. The cloud network infrastructure 66 is a
composition of two and/or more cloud networks 18 (e.g., private 72,
community 74, and/or public 76, etc.) and/or other types of public
and/or private networks (e.g., intranets, etc.) that remain unique entities
but are bound together by standardized or proprietary technology that
enables data and application portability (e.g., cloud bursting for load-
balancing between clouds, etc.)

Cloud software 64 for electronic content retrieval takes full advantage of the cloud paradigm by being service oriented with a focus on statelessness, low coupling, modularity, and semantic interoperability for electronic content retrieval. However, cloud software services 64 can include various states.

Cloud storage of desired electronic content on a cloud computing network includes agility, scalability, elasticity and multi-tenancy. Although a storage foundation may be comprised of block storage or file storage such as that exists on conventional networks, cloud storage is typically exposed to requesters of desired electronic content as cloud objects.

In one exemplary embodiment, the cloud application 30, 30a, 30b, 30c, 30d, offers cloud services for online dynamic diagnosis of respiratory diseases. The application 30, 30a, 30b, 30c, 30d, 30e, 30f offers the cloud computing Infrastructure 66, 68 as a Service 62 (IaaS), including a cloud software infrastructure service 62, the cloud Platform 70, 71 as a Service 62 (PaaS) including a cloud software platform service 62 and/or offers Specific cloud software services as a Service 64 (SaaS) including a specific cloud software service 64 for online dynamic diagnosis of respiratory diseases services. The IaaS, PaaS and SaaS include one or more of cloud services 62 comprising networking, storage, server network device, virtualization, operating system, middleware, run-time, data and/or application services, or plural combinations thereof, on the cloud communications network 18.

FIG. 5 is a block diagram 80 illustrating an exemplary cloud storage object 82. One or more server network devices (e.g., 20, 22, 24, 26, etc.) store portions 13′, 15′ of the electronic message content 13, 15 (e.g., SMS, MMS, RCS, DM, IM, social media, network protocol, telephony, etc., etc.) as cloud storage objects 82 (FIG. 5) as is described herein.

The cloud storage object 82 includes an envelope portion 84, with a header portion 86, and a body portion 88. However, the present invention is not limited to such a cloud storage object 82 and other cloud storage objects and other cloud storage objects with more, fewer or other portions can also be used to practice the invention.

The envelope portion 84 uses unique namespace Uniform Resource Identifiers (URIs) and/or Uniform Resource Names (URNs), and/or Uniform Resource Locators (URLs) unique across the cloud communications network 18 to uniquely specify, location and version information and encoding rules used by the cloud storage object 82 across the whole cloud communications network 18. For more information, see IETF RFC-3305, Uniform Resource Identifiers (URIs), URLs, and Uniform Resource Names (URNs), the contents of which are incorporated by reference.

The envelope portion 84 of the cloud storage object 82 is followed by a header portion 86. The header portion 86 includes extended information about the cloud storage objects such as authorization and/or transaction information, etc.

The body portion 88 includes methods 90 (i.e., a sequence of instructions, etc.) for using embedded application-specific data in data elements 92. The body portion 88 typically includes only one portion of plural portions of application-specific data 92 and independent data 94 so the cloud storage object 82 can provide distributed, redundant fault tolerant, security and privacy features described herein.

Cloud storage objects 82 have proven experimentally to be a highly scalable, available and reliable layer of abstraction that also minimizes the limitations of common file systems. Cloud storage objects 82 also provide low latency and low storage and transmission costs.

Cloud storage objects 82 are comprised of many distributed resources, but function as a single storage object, are highly fault tolerant through redundancy and provide distribution of desired electronic content across public communication networks 76, and one or more private networks 72, community networks 74 and hybrid networks 78 of the cloud communications network 18. Cloud storage objects 82 are also highly durable because of creation of copies of portions of desired electronic content across such networks 72, 74, 76, 78 of the cloud communications network 18. Cloud storage objects 82 includes one or more portions of desired electronic content and can be stored on any of the 72, 74, 76, 78 networks of the cloud communications network 18. Cloud storage objects 82 are transparent to a requester of desired electronic content and are managed by cloud applications 30, 30a, 30b, 30c, 30d.

In one embodiment, cloud storage objects 82 are configurable arbitrary objects with a size up to hundreds of terabytes, each accompanied by with a few kilobytes of metadata. Cloud objects are organized into and identified by a unique identifier unique across the whole cloud communications network 18. However, the present invention is not limited to the cloud storage objects described, and more fewer and other types of cloud storage objects can be used to practice the invention.

Cloud storage objects 82 present a single unified namespace or object-space and manages desired electronic content by user or administrator-defined policies storage and retrieval policies. storage objects include Representational state transfer (REST), Simple Object Access Protocol (SOAP), Lightweight Directory Access Protocol (LDAP) and/or Application Programming Interface (API) objects and/or other types of cloud storage objects. However, the present invention is not limited to the cloud storage objects described, and more fewer and other types of cloud storage objects can be used to practice the invention.

REST is a protocol specification that characterizes and constrains macro-interactions storage objects of the four components of a cloud communications network 18, namely origin servers, gateways, proxies and clients, without imposing limitations on the individual participants.

SOAP is a protocol specification for exchanging structured information in the implementation of cloud services with storage objects. SOAP has at least three major characteristics: (1) Extensibility (including security/encryption, routing, etc.); (2) Neutrality (SOAP can be used over any transport protocol such as HTTP, SMTP or even TCP, etc.), and (3) Independence (SOAP allows for almost any programming model to be used, etc.)

LDAP is a software protocol for enabling storage and retrieval of electronic content and other resources such as files and devices on the cloud communications network 18. LDAP is a “lightweight” version of Directory Access Protocol (DAP), which is part of X.500, a standard for directory services in a network. LDAP may be used with X.509 security and other security methods for secure storage and retrieval. X.509 is public key digital certificate standard developed as part of the X.500 directory specification. X.509 is used for secure management and distribution of digitally signed certificates across networks.

An API is a particular set of rules and specifications that software programs can follow to communicate with each other. It serves as an interface between different software programs and facilitates their interaction and provides access to dynamic diagnosis of respiratory diseases processing services in a cloud or non-cloud environment. In one embodiment, the API for online dynamic diagnosis of respiratory diseases services is available to network devices 12, 14, 16, 20, 22, 24, 26, 29, 31, 33, 98-104 and networks 18, 18′. However, the present invention is not limited to such an embodiment and other embodiments can be used to practice the invention.

### Wearable Devices

Wearable technology” and/or “wearable devices” are clothing and accessories incorporating computer and advanced electronic technologies. Wearable network devices provide several advantages including, but not limited to: (1) Quicker access to notifications. Important and/or summary notifications are sent to alert a user to view the whole message. (2) Heads-up information. Digital eye wear allows users to display relevant information like directions without having to constantly glance down; (3) Always-on Searches. Wearable devices provide always-on, hands-free searches; and (4) Recorded data and feedback. Wearable devices take telemetric data recordings and providing useful feedback for users for exercise, health, fitness, etc. activities.

FIG. 6 is a block diagram with 96 illustrating wearable devices. The wearable devices include one or more processors and include, but are not limited to, wearable digital glasses 98, clothing 100, jewelry 102 (e.g., smart rings, smart earrings, etc.) and/or watches 104. However, the present invention is not limited to such embodiments and more, fewer and other types of wearable devices can also be used to practice the invention.

In one specific embodiment, the application 30, 30a, 30b, 30c, 30d, 30e, 30f interacts with wearable devices 98-104 automatic online dynamic diagnosis of respiratory diseases services the methods described herein. However, the present invention is not limited this embodiment and other embodiments can also be used to practice the invention.

### Artificial Intelligence (AI) and Big Data

“Artificial intelligence” (AI), also known as machine intelligence (MI), is intelligence demonstrated by machines, in contrast to the natural intelligence (NI) displayed by humans and other animals. AI research is defined as the study of “intelligent agents.” Intelligent agents are any software application or hardware device that perceives its environment and takes actions that maximize its chance of successfully achieving its goals. Colloquially, the term “artificial intelligence” is applied when a machine mimics “cognitive” functions that humans associate with human brains, such as learning, problem solving and comparing large number of data points.

In one embodiment, the present invention uses one or more AI methods including, but are not limited to, AI knowledge-based methods 30c for dynamic diagnosis of respiratory diseases services. In one embodiment, the AI application 30c includes AI for dynamic diagnosis of respiratory diseases. However, the present invention is not limited to such an embodiment and more, fewer and/or other AI methods with more and/or different AI functionality can be used to practice the invention.

In one embodiment, SaaS 64 includes and AI application 30c with the AI methods described herein. In another embodiment, the AI application 30c is a standalone application. However, the present invention is not limited to such an embodiment, and the AI application 30c can be provided in other than the SaaS 64.

“Big Data” refers to the use of predictive analytic methods that extract value from data, and to a particular size of data set. The quantities of data used are very large, at least 100,000 data points and more typically 500,000 to 1 Million+ data points. Analysis of Big Data sets are used to find new correlations and to spot trends. In one embodiment, SaaS 64 includes and Big Data application 30d with the Big Data described herein.

In one embodiment, the AI methods described herein collect data information to create and store (e.g., in cloud storage object 82, etc.) a Big Data that is used to analyze trends find new correlations and to spot trends. However, the present invention is not limited to such an embodiment and the AI methods described herein can be used without Big Data sets.

In one embodiment, the AI application 30c includes an Artificial Intelligence (AI) technology stack 61 (FIG. 3) is used within AI application 30c (FIG. 1) and includes complete, end-to-end solution that consists of hardware, software, and tools that facilitate the development and deployment of AI applications. The AI technology stack includes specialized tools to support the building of AI models that enable machine learning and deep learning.

The AI technology stack 61 includes, but is not limited to, four foundational layers: an AI application layer 63 (FIG. 3) an AI model layer 65, an AI data layer 67, and an AI infrastructure layer 69.

The AI application layer 63 of the AI tech stack includes any software, user interfaces, and accessibility features that enable users to interact with the underlying AI models and the datasets that power an AI solution. For example, browser-based interfaces allow users to send questions to a Generative AI model like CHATGPT, or a data analytics suite including Predictive AI to provide visualizations in the form of graphs and charts to help users understand the AI model's results.

Generative AI 73 (GenAI, or GAI) is a subset of AI that uses Generative AI models to produce new text, images, videos, and/or other forms of data.

Predictive AI 75 (PredAI or PAI) is also a subset of AI that uses Predictive AI models, machine learning and statistical analysis to forecast future events. Machine learning is a field of study in AI concerned with the development and study of statistical algorithms that can learn from data and generalize to unseen data, and thus perform tasks without explicit instructions.

In one embodiment the AI application 30c and/or the AI technology stack 61, includes but is not limited to, a Generative AI component 73 and/or a Predictive AI component 75. In one embodiment, the Generative AI component 73 and the Predictive AI component 75 are standalone components of the AI application 30c. In another embodiment, the Generative AI component 73 the Predictive AI component 75 are included as layers in the AI technology stack 61. However, the present invention is not limited to such embodiments and other embodiments and/or other combinations can be used to practice the invention.

The AI model layer 65 of the AI technology stack 61 is where AI models are developed, trained, and optimized. AI models are developed using a combination of AI frameworks, toolsets, and libraries and are subsequently trained on vast amounts of data to help refine their decision-making processes.

The AI data layer 67 layer focuses on dataset collection, storage, and management, interfacing with and enabling all the other layers. Data from this layer is fed to the AI model layer 65, new data from the AI application layer 63 is captured here for future model analysis, and the AI infrastructure layer 69 provides the resources needed to scale, secure, and reliably process the data.

The AI infrastructure layer 69 of the AI technology stack 61 includes all hardware and compute resources needed to run AI models in the AI models layer 65 and any user-facing software. This can include enterprise data centers, cloud 18 or non-cloud 18′ server network devices 20, 22, 24, 26, client/target network devices 12, 14, 16, 31, 98-104, etc.

In one embodiment, the AI application 30c, includes but is not limited to, plural different AI agents. However, the present invention is not limited to such an embodiment and other embodiments, with more, fewer and/or other types of AI can be used to practice the invention.

In one exemplary embodiment, the plural AI agents 30c1, 30c2, 30c3 (FIG. 1), from AI application 30c, include, but are not limited to, the functionality included in Table 4.

TABLE 4
1. AI-Powered Generative AI Conversational Agent 30c1 for
Hyper-Personalized Patient 35, 35′ Interactions
Description: Using Generative AI 73 to create advanced methods and
conversational agents for patients. These agents can analyze patient data, past
interactions, and real-time context to provide tailored responses, product
recommendations, and support.
Generative AI 73 AI Technologies:
Generative AI 73 Hosted AI Models: Pre-trained models hosted on a
platform for general use cases (e.g., sentiment analysis, intent recognition).
Generative AI 73 Private Trained Models: Custom AI models fine-
tuned on proprietary patient data to ensure brand-specific tone, compliance, and
accuracy.
Generative AI 73 Large Language Models (LLMs): Integration with
LLMs like GPT or proprietary LLMs for natural language understanding and
generation.
Generative AI 73 Multimodal AI: Combining audio, text, image, and
video analysis to enable richer interactions (e.g., analyzing a product image sent by a
patient and suggesting solutions).
2. AI-Powered Predictive AI Analytics Agent 30c2 for Proactive
Patient Engagement
Description: Using Predictive AI 75 to analyze patient behavior,
preferences, and historical data to predict future needs and proactively engage patients
via messaging. For example, predicting when a patient might need medical
information, a medical referral and sending timely, context-aware messages.
Predictive AI 75 AI Technologies:
Predictive AI 75 Public AI Models: Leveraging publicly available
models for Predictive analytics (e.g., time-series forecasting models).
Predictive AI 75 Private Trained Models: Custom models trained on
proprietary medical data to predict patient behavior with high accuracy.
Predictive AI 75 Reinforcement Learning: AI systems that learn
optimal engagement strategies over time by analyzing patient responses.
Predictive AI 75 Edge AI: Deploying lightweight AI models on
devices to enable real-time predictions without relying on cloud infrastructure.
3. AI-Powered Experience Builder Agent 30c3 for Analyzing
Proactive Patient Experiences
Description: Using AI to power the AI platform that enables
businesses to analyze, build, customize, and deploy patient experiences using APIs,
drag-and-drop workflows, and no-code/low-code tools. This platform allows medical
facilities to create AI-enhanced chatbots, automate workflows, and integrate
messaging with their existing online medical billing systems and online patient
medical information systems (e.g., MYCHART ®, etc.)
AI Technologies:
Hosted AI Models: Pre-trained models for natural language processing
(NLP), intent recognition, and sentiment analysis, accessible via APIs.
Private Trained Models: Custom AI models that businesses can train
on their own data to create brand-specific conversational experiences.
Generative AI 73: Leveraging LLMs to generate conversational
scripts, FAQs, and responses dynamically.
Predictive AI 75: Analyzing patient behavior, preferences, and
historical data to predict future needs and proactively engage patient via messaging.
Workflow Automation AI: AI systems that automate the creation of
complex workflows, such as routing messages to the right medical department or
triggering actions based on patient input.
Explainable AI (XAI): Tools that provide transparency into how AI
models generate responses, ensuring trust and compliance.

### Short Message Service Messaging

Short Message Service (SMS) is an electronic text messaging service component of phone, Web, or mobile communication systems. It uses standardized communications protocols to allow fixed line or mobile phone devices to exchange short text messages.

SMS messages were defined in 1985 as part of the Global System for Mobile Communications (GSM) series of standards as a means of sending messages of up to 160 characters to and from GSM mobile handsets. Though most SMS messages are mobile-to-mobile text messages, support for the service has expanded to include other mobile technologies as well as satellite and landline networks.

The SMS Internet Engineering Task Force (IETF) Request for Comments (RFC) 5724, ISSN: 2070-1721, 2010, is incorporated herein by reference.

### Direct and Instant Messages

A “direct message” (DM) is a private form of communication between social media users that is only visible to the sender and recipient(s). INSTAGRAM, TWITTER, FACEBOOK and other platforms, allow for direct messages between their users, with varying restrictions by platform.

An “instant message” (IM) is a type of online chat allowing real-time text transmission over the Internet or another computer network. Messages are typically transmitted between two or more parties, when each user inputs text and triggers a transmission to the recipient, who are all connected on a common network.

### Multimedia Messaging Service (MMS)

Multimedia Messaging Service (MMS) is a standard way to send messages that include multimedia content to and from a mobile phone over a cellular network. Users and providers may refer to such a message as a PXT, a photograph message, and/or a multimedia message.

The MMS Internet Engineering Task Force (IETF) Request for Comments (RFC) 4355 and 4356, are incorporated herein by reference.

### Rich Communication Suite (RCS)

Rich Communications Suite/Rich Communications System (RCS) is a communication protocol between mobile telephone carriers, between phones and carriers, and between individual devices aiming at replacing SMS messages with a message system that is richer, provides phonebook polling (e.g., for service discovery, etc.), and can transmit in-call multimedia. It is also marketed under the names of Advanced Messaging, Advanced Communications, Chat, joyn, Message+ and SMS+. RCS is also a communication protocol available for device-to-device (D2D) exchanges without using a telecommunications carrier for devices that are in close physical proximity (e.g., between two IoT devices, smart phones, smart phone and electronic tablet, etc.)

One advantage RCS Messaging has over SMS is that RCS enables users to send rich, verified messages including photos, videos and audio messages, group messages, read receipts, indicators to show other users are typing a message, carousel messages, suggested chips, chat bots, barcodes, location integration, calendar integration, dialer integration, and other RCS messaging features. RCS messaging includes person-to-person (P2P), application-to-person (A2P), application-to-application (A2A), application-to-device (A2D) and/or device-to-device (D2D) messaging.

The RCS Interworking Guidelines Version 14.0, 13 Oct. 2017, GSM Association, Rich Communication Suite RCS API Detailed Requirements, version 3.0, Oct. 19, 2017, Rich Communication Suite 8.0 Advanced Communications Services and Client Specification Version 9.0, 16 May 2018, RCS Universal Profile Service Definition Document Version 2.2, 16 May 2018, and Rich Communication Suite Endorsement of OMA CPM 2.2 Conversation Functions Version 9.0, 16 Oct. 2019, are all incorporated herein by reference.

The Rich Communication Suite-Enhanced (RCS-e) includes methods of providing first stage interoperability among Mobile Network Operators (MNOs). RCS-e is a later version of RCS which enables mobile phone end users to use instant messaging (IM), live video sharing and file transfer across any device on any MNO.

The RCS functionality of the present invention includes, but is not limited to, one and two-way, rich, verified, multimedia messages including photos, videos and audio messages, group messages, read receipts, indicators to show other users are typing a message, predefined quick-reply suggestions, rich cards, carousels, action buttons, maps, click-to-call, calendar integration, geo-location, etc. The RCS functionality also includes RCS emulators and/or thin RCS applications that provide full and/or selected features of available RCS functionality. The RCS message application 30a and the RCS interoperability application 30b provides full and/or partial RCS functionality including, but not limited to, RCS-e functionality. However, the present invention is not limited to such embodiments.

### Automated Dynamic Diagnosis of Respiratory Diseases

The method and system described herein addresses a critical gap in healthcare diagnostics by providing a portable, accessible, and affordable solution for respiratory disease detection. Its implementation significantly improves early detection rates, reduces misdiagnoses, and facilitate prompt treatment of respiratory diseases. Moreover, it includes an Artificial Intelligence (AI) based system that can be used by a patient in person at urgent care center, medical office, hospital, etc. and is also integrated into remote telehealth and/or telemedicine platforms for remote use, expanding access to health care for patients.

Early detection of diseases from vocal and cough sounds is critical, particularly for respiratory illnesses. Traditional diagnostic tools are often time-consuming and out of reach. This the method and system described herein provide a fast, non-invasive diagnostic tool that leverages machine learning to analyze audio signals, specifically focusing on cough sounds and voice as biomarkers.

Audio recordings of coughs and voice sounds are collected and analyzed to detect biomarkers that serve as indicators of various diseases respiratory illnesses. Audio signals are transformed into waveform graphs and spectrograms for distinct processing pathways. A final diagnostic report is prepared including one or more cough types and one or more disease categories and the calibrated probabilities for a patient.

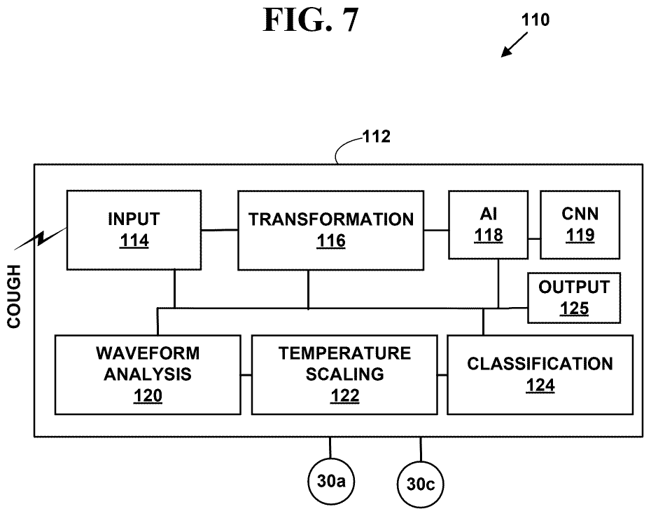

FIG. 7 is a block diagram 110 illustrating an exemplary dynamic diagnosis of respiratory disease system 112.

The system 112, includes, but is not limited to, an input module 114 to capture audio data including voice data and cough data and demographic data for a patient, a transformation module 116 to generate a waveform and a spectrogram from the captured audio data, an Artificial Intelligence (AI) module 118 with a Convolutional Neural Network (CNN) module 119 to analyze the spectrogram and extract disease-specific spectral features, a waveform analysis module 120 to calculate Zero Rate Crossing (ZCR), Chroma Features, Spectral Contrast, Magnitude, Root Mean Square (RMS), and Short-time Fourier Transform (STFT) as additional cough or disease biomarkers, a temperature scaling module 122 to calibrate the classifier's probability outputs, a classification module 124 to combine the CNN and waveform features, applying dense layers to classify disease types based on cough and disease biomarkers, and an output module 125 for displaying exemplary output information including cough biomarker analysis, a disease and/or illness based on the voice data and cough data and/or a probability of the disease and/or illness based on the voice data and cough data. However, the present invention is not limited to such an embodiment and more, fewer and/or other modules can be used to practice the invention.

A waveform includes, but is not limited to, a graphic representation of a shape of a wave over a time period that indicates its characteristics, including frequency and amplitude and is also called a waveshape.

A spectrogram, includes, but is not limited to, a visual representation of the spectrum of frequencies of a signal as it varies with time. When applied to an audio signal, spectrograms are also called sonographs, voiceprints, and/or voicegrams.

The Artificial Intelligence (AI) module 118, includes, but is not limited to, an Artificial Intelligence (AI) application 30c that includes generative AI 73 methods, models, large language models (LLMs) and/or predictive AI 75 methods, models, large language models (LLMs) (See, Table 4, etc.). The AI module 118 uses the CNN module 119 to analyze the spectrogram and extract disease-specific spectral features. However, the present invention is not limited to such embodiments and other embodiments may be used to practice the invention.

A Convolutional Neural Network (CNN) module 119, includes, but is not limited to, a type of artificial neural network that uses deep learning to analyze visual data and identify patterns in images. A CNN module 119 includes a regularized type of feed-forward neural network that learns features by itself via filter or kernel optimization.

In one embodiment, the CNN module 119, includes, but is not limited to, the CNN module consists of multiple convolutional layers with progressively increasing filter sizes and Max Pooling, enabling deep extraction of features relevant to cough type and disease detection. However, the present invention is not limited to such an embodiment and other types of CNN can be used to practice the invention.

Max Pooling, includes, but is not limited to, a technique used in CNN modules 119 used to reduce spatial dimensions of an input image while retaining the most important information. Max Pooling divides an input image into non-overlapping regions, calculates a maximum value for each region, and uses those maximum values to create a down sampled output.

Zero Crossing Rate (ZCR), includes but is not limited to, a measurement of how often an audio waveform crosses a zero axis. It includes a rate at which a signal changes from positive to zero to negative or from negative to zero to positive.

Chroma Features, includes, but is not limited to, capturing harmonic and melodic characteristics of audio information including voice information and cough information.

Spectral Contrast, includes, but is not limited to, considering a spectral peak, a spectral valley, and their difference in each frequency sub-band in audio information including voice information and cough information.

Magnitude, includes, but is not limited to, a relationship between how often an event occurs, its frequency and its intensity or strength, its magnitude.

Root mean square (RMS), includes, but is not limited to, a root mean square of a set of numbers is a square root of a set's mean square.

Short-time Fourier Transform (STFT), includes, but is not limited to, a Fourier-related transform used to determine a sinusoidal frequency and phase content of local sections of a frequency signal as it changes over time. In practice, the procedure for computing STFTs is to divide a longer time signal into shorter segments of equal length and then compute the Fourier transform separately on each shorter segment. This reveals the Fourier spectrum on each shorter segment. The changing spectra is plotted as a function of time on a spectrogram.

Temperature scaling, is a mathematical technique that includes, but is not limited to, dividing a logits vector by a learned scalar parameter. The scalar parameter is learned on a validation set, where T in temperature scaling is chosen to minimize negative log likelihood. This controls the sharpness or flatness of a predicted probability distribution. Temperature scaling improves a confidence of a neural network (e.g., the AI 118 and CNN 119, etc.) after training. The calibrated probabilities then approximately represent a confidence score of model predictions (e.g., a confidence interval 136 for type of cough discovered 134, etc.).

In one embodiment, the temperature scaling module 122, includes but is not limited to, applying post-processing to the classifier logits to enhance calibration accuracy, providing reliable probability estimates of types of of respiratory diseases.

In one embodiment, the dynamic diagnosis respiratory disease diagnostic application includes a software application, a firmware application or a hardware application. However, the present invention is not limited to such embodiments and other embodiments may be used to practice the invention.

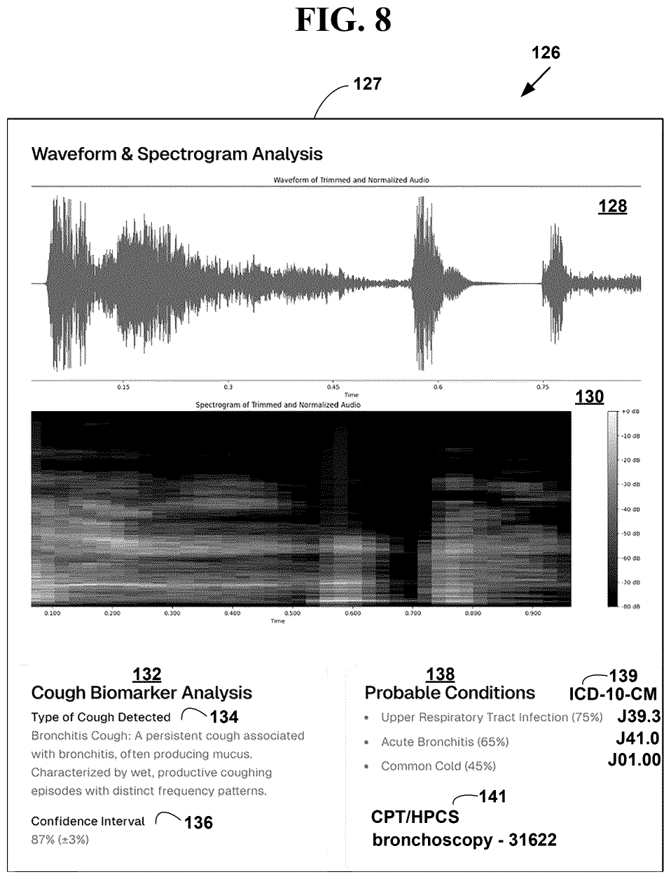

FIG. 8 is a block diagram 126 illustrating outputs from an exemplary dynamic diagnosis of respiratory diseases system 112.

FIG. 8 includes an actual output screen 127 created and displayed via the server respiratory disease diagnosis application 30a on the server network device 20, 22, 24, 26.

FIG. 8 includes an exemplary waveform component 128, created from audio data collected from a patient 35, 35′ for a cough, a spectrogram component 130, created from the audio data collected from a patient 35, 35′ from a cough, a cough biomarker analysis component 132 including a type of cough detected 134, a confidence interval 136 and a probable condition component 138 including physical conditions associated with the cough and medical diagnosis codes 139 and/or medical billing codes 141. However, the present invention is not limited to such an embodiment and more, fewer and/or other components can be used to practice the invention.

A spectrogram is analyzed using the AI module 118 and the Convolutional Neural Network (CNN) module 119 to extract spectral features, while the waveform is processed to calculate key features, including Zero Crossing Rate (ZCR), Chroma Features, Spectral Contrast, Magnitude, Root Mean Square (RMS), and Short-Time Fourier Transform (STFT). These features, when used collectively, support biomarkers for cough and disease classification.

Temperature scaling is applied to improve model calibration, ensuring more accurate and reliable prediction probabilities of cough types and disease, especially important in medical applications where diagnostic certainty is critical.

FIG. 8 illustrates a probable condition component 138 listing the probable diagnosis of the couch as: (1) upper respiratory tract infection 75%; (2) acute bronchitis 65% and (3) common cold 45%.

Medical diagnosis codes 139 are alphanumeric codes used to classify and code diseases and medical conditions for healthcare purposes like billing, record-keeping, and statistics. They are used identify a patient's 35, 35′ disease, condition, or symptom. The current system in the U.S. is the ICD-10-CM (International Classification of Diseases, Tenth Revision, Clinical Modification), the contents of which are incorporated herein by reference, which uses codes that are alphanumeric, have between three and seven characters, and can include a decimal point for more specificity.

FIG. 8 illustrates actual ICD-10-CM medical diagnosis codes 139 automatically generated for upper respiratory tract infection, J39.3, acute bronchitis, J41.0, common cold respiratory, J01.00. However, the present invention is not limited to this embodiment and the invention can be practiced without automatically generating and/or displaying any medical diagnosis codes 139.

Medical billing codes 141 are alphanumeric codes used on healthcare claims to identify medical procedures performed on a patient 35, 35′ and medical services provided to a patient 35, 35′. The most common medical diagnosis billing codes also include Current Procedural Terminology (CPT)/Healthcare Common Procedure Coding System (HCPCS) codes, the contents of which are incorporated by reference. These codes describe the medical services or procedures provided, such as an office visit or a specific test, which are billed alongside the medical diagnosis codes.

In one embodiment, no medical billing codes 141 are automatically generated because no medical services or medical procedures are provided by medical personnel. If medical services or medical procedures are provided after the respiratory disease diagnosis, then medical billing codes 141 are automatically generated. However, the present invention is not limited to this embodiment and the invention can be practiced without automatically generating any medical billing codes 141.

FIG. 8 illustrates an actual CPT/HCPCS code for a bronchoscopy, 31622. A bronchoscopy is a minimally invasive procedure where a flexible tube with a light and camera called a bronchoscope is inserted through the nose or mouth to examine the lungs and airways. A procedure that may be completed on a patient 35, 35′ that was diagnosed with acute bronchitis, J41.0. However, the present invention is not limited to this embodiment and the invention can be practiced without automatically generating and/or displaying any medical billing codes 141.

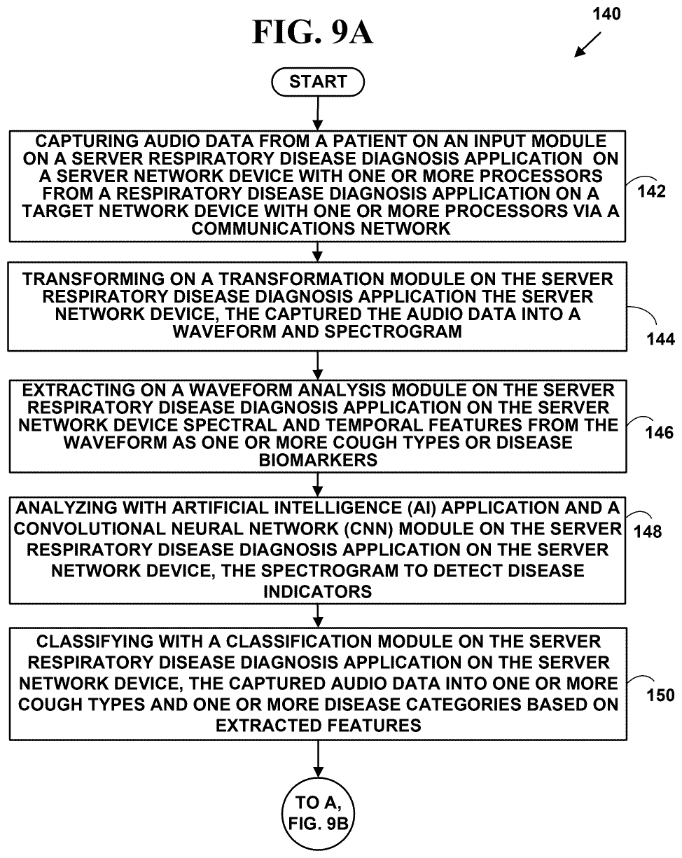

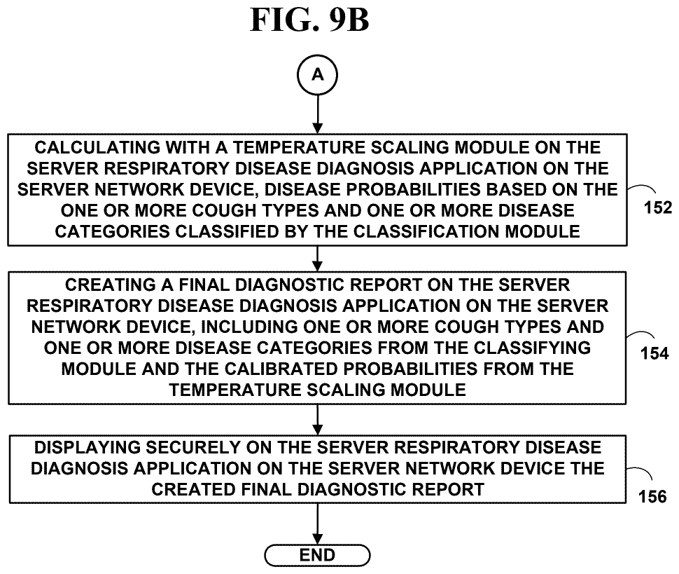

FIGS. 9A and 9B are a flow diagram illustrating a Method 140 for dynamic diagnosis of respiratory diseases.

In FIG. 9A at Step 142, capturing audio data from a patient on an input module on a server respiratory disease diagnosis application on a server network device with one or more processors from a respiratory disease diagnosis application on a target network device with one or more processors via a communications network. At Step 144, transforming on a transformation module on the server respiratory disease diagnosis application on the server network device, the captured the audio data into a waveform and spectrogram. At Step 146 extracting on a waveform analysis module on the server respiratory disease diagnosis application on the server network device, spectral and temporal features from the waveform as one or more cough types or disease biomarkers. At Step 148, analyzing with and Artificial Intelligence (AI) module and a Convolutional Neural Network (CNN) module on the server respiratory disease diagnosis application on the server network device, the spectrogram to detect disease indicators. At Step 150, classifying with a classification module on the server respiratory disease diagnosis application on the server network device, the captured audio data into one or more cough types and one or more disease categories based on extracted features and calibrated probabilities; In FIG. 9B at Step 152, calculating with a temperature scaling module, on the server respiratory disease diagnosis application on the server network device, disease probabilities based on the one or more cough types and one or more disease categories classified by the classification module. At Step 154, creating a final report on the server respiratory disease diagnosis application on the server network device including one or more cough types and one or more disease categories from the classifying module and the calibrated probabilities from the temperature module At Step 156, displaying securely on the server respiratory disease diagnosis application on the server network device the created final diagnostic report.

Method 140 is illustrated with an exemplary embodiment. However, the present invention is not limited to this exemplary embodiment and other embodiments can be used to practice the invention.

In such an exemplary embodiment, in FIG. 9A at Step 142, capturing audio data 13,15 including cough data and/or voice data from a patient 35, 35′ on an input module 114 on a server respiratory disease diagnosis application 30a on a server network device 20 with one or more processors from a respiratory disease diagnosis application 30 on a target network device 12, 14, 16, 29, 31, 33, 98-104 with one or more processors via a communications network 18, 18′.

At Step 144, transforming on a transformation module 116 on the server respiratory disease diagnosis application 30a, on the server network device 20 the captured the audio data 13, 15 into a waveform 128 and spectrogram 130.

At Step 146, extracting on a waveform analysis module 118 on the server respiratory disease diagnosis application 30a on the server network device 20, spectral and temporal features from the waveform 128 as one or more cough types or disease biomarkers.

At Step 148, analyzing with an Artificial Intelligence (AI) module 118 including AI application 30c and a Convolutional Neural Network (CNN) module 119 on the server respiratory disease diagnosis application 30a on the server network device 20, the spectrogram 130 to detect disease indicators.

In one embodiment, the Artificial Intelligence (AI) application 30c includes generative AI 73 methods, models, large language models (LLMs) and/or predictive AI 75 methods, models, large language models (LLMs) (See, Table 4, etc.). However, the present invention is not limited to such embodiments and other embodiments may be used to practice the invention.

At Step 150, classifying with a classification module 124 on the server respiratory disease diagnosis application 30a on the server network device 20, the captured audio data into one or more cough types and one or more disease categories based on extracted features.

In FIG. 9B, at Step 152, calculating with a temperature scaling module 122 on the server respiratory disease diagnosis application 30a on the server network device 20, disease probabilities based on the one or more cough types and one or more disease categories classified by the classification module 124.

At Step 154, creating a final diagnostic report 13d, 15d on the server respiratory disease diagnosis application 30a on the server network device 20, including one or more cough types and one or more disease categories from the classifying module 124 and the calibrated probabilities from the temperature scaling module 122.

At Step 156, displaying securely on the server respiratory disease diagnosis application 30a on the server network device 20, 22, 24, 26 the created final diagnostic report 13d, 15d.

The created final diagnostic report 13d, 15d is securely displayed using one or more of the encryption and/or security methods described herein to protect privacy of the patent 35, 35′ as is required by the Health Insurance Portability and Accountability Act (HIPAA) of 1996, 42 U.S.C. § 1320d et al., 45 C.F.R. Parts 160 and 164. HIPAA is also cited as Public Law 104-191, that is a U.S. federal law that protects patients' 35, 35′ health information and gives them rights over their health records.

In one embodiment, the final diagnostic report 13d, 15d is also securely sent to and/or displayed on the respiratory disease diagnosis application 30 on the target network device via the communications network 18, 18′. The final diagnostic report 13d, 15d is securely sent using with one or more secure messages using one or more of the encryption and/or security methods described herein also to protect patient 35, 35′ privacy. However, the present invention is not limited to such embodiments and other embodiments can be used to practice the invention.

In one embodiment, the output module automatically generates medical diagnosis codes 139 and/or medical billing codes 141 that are displayed at Step 154 and included in in the final diagnostic report 13d, 15d based on one or more cough types and one or more disease categories in the final diagnostic report 13d, 15d. However, the present invention is not limited to such an embodiment and other embodiments, with and/or without medical diagnosis codes and/or medical diagnosis billing codes can be used to practice the invention.

In one embodiment, the server respiratory disease diagnosis application 30a includes a SaaS 64 on a cloud communications network 18. However, the present invention is not limited to such embodiments and other embodiments can be used to practice the invention.

In one embodiment, Method 140 further includes sending an electronic link for the created final diagnostic report 13d, 15d stored on the server network device 20 from the server respiratory disease diagnosis application 30a on the server network device 20 to the respiratory disease diagnosis application 30 on the target network device 12, 14, 16, 29, 31, 33, 98-104 via the communications network 18, 18′. However, the present invention is not limited to such embodiments and other embodiments can be used to practice the invention.

In one embodiment, the electronic link is an HTML, XML, RCS and/or other type of electronic link. However, the present invention is not limited to such embodiments and other embodiments can be used to practice the invention.

In one embodiment, the electronic link is automatically activated. In another embodiment, the electronic link is manually activated with a selection input (e.g., clicking on the link, etc.).

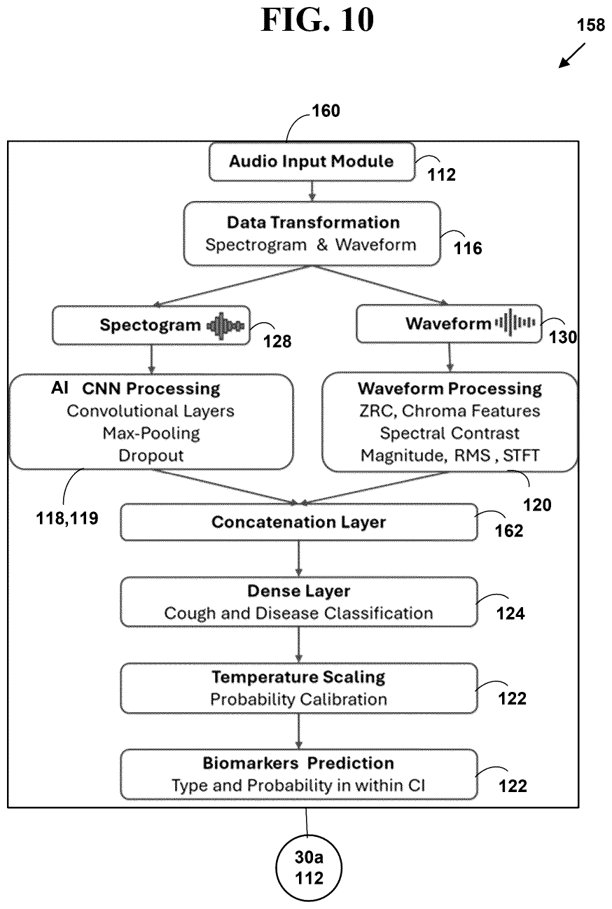

FIG. 10 is a block diagram 160 illustrating an exemplary data flow 158 for the exemplary dynamic diagnosis of respiratory diseases system 10.

In FIG. 10, additional data flow details 160 are displayed. FIG. 10 illustrates a concatenation layer 162 to combine results from the AI module 118 and CNN processing module 119 and the waveform analysis module 120. However, the present invention is not limited to such embodiments and other embodiments can be used to practice the invention.

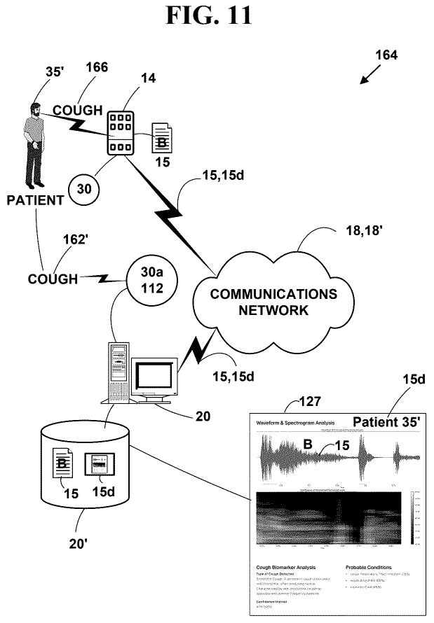

FIG. 11 is a block diagram 164 illustrating an exemplary data flow for the exemplary dynamic diagnosis of respiratory diseases system 10.

In FIG. 11, a patient 35′ coughs 166 in a target network device 16 to create audio data 15. This audio data is processed using dynamic diagnosis of respiratory disease system 112 and Method 140 to obtain final diagnostic report 15d displayed as output screen 127. However, the present invention is not limited to such embodiments and other embodiments can be used to practice the invention.

In another embodiment, the patient 35′ coughs 164′ directly into server respiratory disease diagnosis application 30a on the server network device 20, 22, 24, 26, for example at an urgent care facility, medical office, hospital, etc. However, the present invention is not limited to such embodiments and other embodiments can be used to practice the invention.

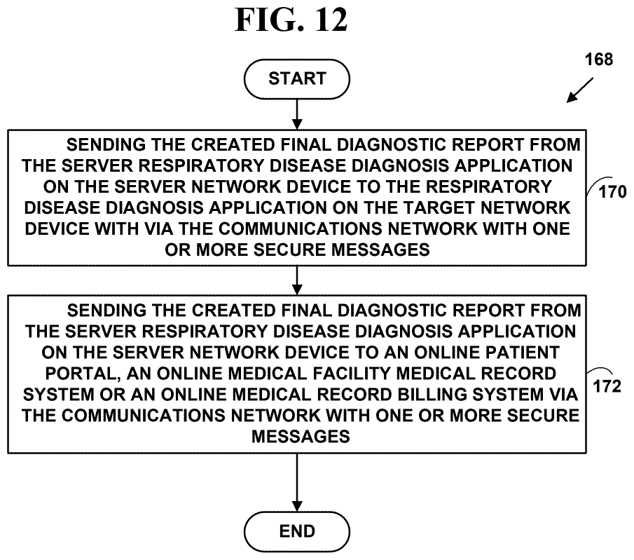

FIG. 12 is a flow diagram illustrating a Method 168 for dynamic diagnosis of respiratory diseases.

In FIG. 12 at Step 170, sending the created final diagnostic report from the server respiratory disease diagnosis application on the server network device to the respiratory disease diagnosis application on the target network device with via the communications network with one or more secure messages. At Step 172, sending the created final diagnostic report from the server respiratory disease diagnosis application on the server network device to an online patient portal, an online medical facility medical record system or an online medical record billing system via the communications network with one or more secure messages.

Method 168 is illustrated with an exemplary embodiment. However, the present invention is not limited to this exemplary embodiment and other embodiments can be used to practice the invention.

In such an exemplary embodiment, in FIG. 12 at Step 170, sending the created final diagnostic report 13d, 15d, from the server respiratory disease diagnosis application 30a on the server network device 20 to the respiratory disease diagnosis application 30 on the target network device 12, 14, 16, 29, 31, 33, 98-104 with via the communications network 18, 18′ with one or more secure messages.

At Step 172, sending the created final diagnostic report 13d, 15d from the server respiratory disease diagnosis application 30a on the server network device 20 to an online patient portal 22, an online medical facility medical record system 24 or an online medical record billing system 26 via the communications network 18, 18′ with one or more secure messages.

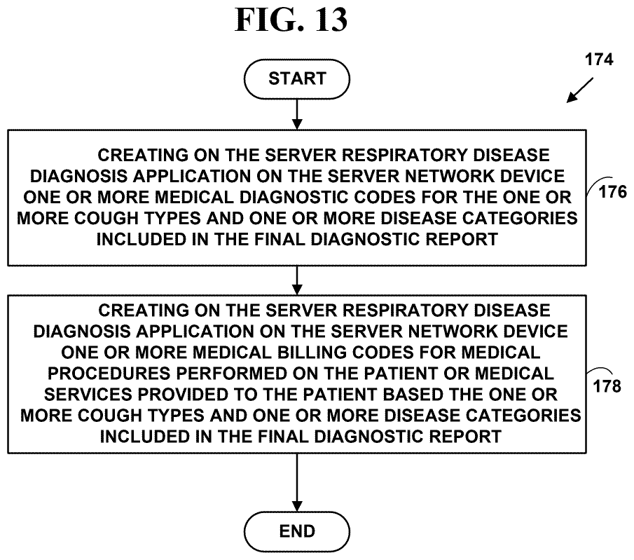

FIG. 13 is a flow diagram illustrating a Method 174 for dynamic diagnosis of respiratory diseases.

In FIG. 13 at Step 176, creating on the server respiratory disease diagnosis application on the server network device one or more medical diagnostic codes for the one or more cough types and one or more disease categories included in the final diagnostic report. At Step 178, creating on the server respiratory disease diagnosis application on the server network device one or more medical billing codes for medical procedures performed on the patient or medical services provided to the patient based the one or more cough types and one or more disease categories included in the final diagnostic report.

Method 174 is illustrated with an exemplary embodiment. However, the present invention is not limited to this exemplary embodiment and other embodiments can be used to practice the invention.

In such an exemplary embodiment, in FIG. 13 at Step 176, creating on the server respiratory disease diagnosis application 30a on the server network device 20, one or more medical diagnostic codes 139 for the one or more cough types and one or more disease categories included in the final diagnostic report 13d, 15d.

At Step 178, creating on the server respiratory disease diagnosis application 30a on the server network device 20 one or more medical billing codes 141 for medical procedures performed on the patient or medical services provided to the patient based the one or more cough types and one or more disease categories included in the final diagnostic report 13d, 15d.

In one embodiment, the server respiratory disease diagnosis application 30a on the server network device 20 provides a telehealth platform to remote patients via the communications network 18, 18′. However, the present invention is not limited to such an embodiment and other embodiments can be used to practice the invention.

A telehealth platform, includes use of electronic information and communications technologies to provide healthcare services, patient 35, 35′ education, and health administration from a distance. Telehealth platforms include virtual visits via phone or video, remote patient 35, 35′ monitoring, and electronic messaging with providers, all using target network devices 12, 14, 16, 29, 31, 33, 98-104.

In one embodiment, the server respiratory disease diagnosis application 30a on the server network device 20 provides a telemedicine platform to remote patients via the communications network 18, 18′. However, the present invention is not limited to such an embodiment and other embodiments can be used to practice the invention.

The telemedicine platform, includes the remote delivery of healthcare services using communications technology, allowing healthcare professionals to diagnose, treat, and monitor patients 35, 35. from a distance. telemedicine platforms encompass a range of medical services provided by video calls, phone consultations, secure messaging, and remote patient 35, 35′ monitoring.

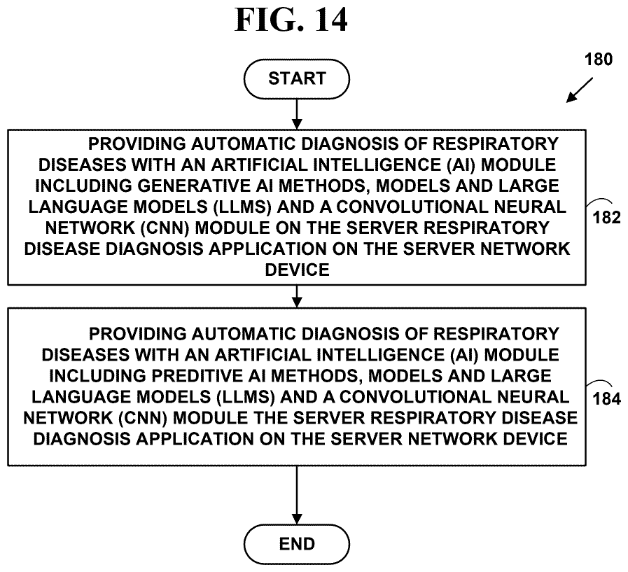

FIG. 14 is a flow diagram illustrating a Method 180 for dynamic diagnosis of respiratory diseases.

In FIG. 14 at Step 182, providing automatic diagnosis of respiratory diseases with an Artificial Intelligence (AI) module including Generative AI methods, models and large language models (LLMs) and a Convolutional Neural Network (CNN) module the server respiratory disease diagnosis application on the server network device. At Step 184, providing automatic diagnosis of respiratory diseases with an Artificial Intelligence (AI) module including Predictive AI methods, models and large language models (LLMs) and a Convolutional Neural Network (CNN) module on server respiratory disease diagnosis application on the server network device.

Method 180 is illustrated with an exemplary embodiment. However, the present invention is not limited to this exemplary embodiment and other embodiments can be used to practice the invention.

In such an exemplary embodiment, In FIG. 14 at Step 182, providing automatic diagnosis of respiratory diseases with an Artificial Intelligence (AI) module 118 including Generative AI 73 methods, models and large language models (LLMs) (Table 4) and a Convolutional Neural Network (CNN) module 119 the server respiratory disease diagnosis application 30a on the server network device 20.

At Step 184, providing automatic diagnosis of respiratory diseases with an Artificial Intelligence (AI) module 118 including Predictive AI 75 methods, models and large language models (LLMs) (Table 4) and a Convolutional Neural Network (CNN) module 119 on the server respiratory disease diagnosis application 30a on the server network device 20.

The methods and system described herein provides an efficient system for disease detection through advanced analysis of cough and voice sounds. It has been determined experimentally, with a large data set, that the final diagnostic report 13d, 15d includes a diagnostic confidence interval (CI) currently in the range of about 83%-96%, depending on the cough type and disease. By extracting and calibrating disease-specific biomarkers, this system offers a non-invasive, reliable, and cost-effective diagnostic aid suitable for medical applications.

The method and system described herein provides new diagnostic tools to transform respiratory healthcare by providing easy access to the diagnostic tools and reducing the economic strain on healthcare systems. In the context of public health emergencies, such as the COVID-19 pandemic, it also serves as a valuable diagnostic tool for large-scale screening and monitoring of a large number of patients.

A method and system for biomarker detection using artificial analysis (AI) with a Convolutional Neural Network (CNN) of cough and voice sounds. Audio recordings of coughs and voice sounds are collected from a patient either in-person or remotely via telemedicine. The collected audio signals are transformed into waveform graphs and spectrograms for distinct processing pathways. AI CNN methods are used to classified cough types and detect respiratory diseases based on non-invasive acoustic signals. A final diagnostic report is prepared including one or more cough types and one or more disease categories and the calibrated probabilities for the patient. By analyzing the unique sound characteristics of coughs underlying respiratory conditions are accurately identified in real-time, offering a cost-effective and scalable diagnostic tool.

of cough and voice sounds is present herein. Audio recordings of coughs and voice sounds are collected from a patient either in-person or remotely. The collected audio signals are transformed into waveform graphs and spectrograms for distinct processing pathways. AI methods are used to classified cough types and detect respiratory diseases based on non-invasive acoustic signals. By analyzing the unique sound characteristics of coughs underlying respiratory conditions are accurately identified in real-time, offering a cost-effective and scalable diagnostic tool.

It should be understood that the architecture, programs, processes, methods and systems described herein are not related or limited to any particular type of computer or network system (hardware or software), unless indicated otherwise. Various types of specialized computer systems may be used with or perform operations in accordance with the teachings described herein.

In view of the wide variety of embodiments to which the principles of the present invention can be applied, it should be understood that the illustrated embodiments are exemplary only, and should not be taken as limiting the scope of the present invention. For example, the steps of the flow diagrams may be taken in sequences other than those described, and more or fewer elements may be used in the block diagrams.

While various elements of the preferred embodiments have been described as being implemented in software, in other embodiments hardware or firmware implementations may alternatively be used, and vice-versa.

The claims should not be read as limited to the described order or elements unless stated to that effect. In addition, use of the term “means” in any claim is intended to invoke 35 U.S.C. § 112, paragraph 6, and any claim without the word “means” is not so intended.

Therefore, all embodiments that come within the scope and spirit of the proceeding described and equivalents thereto are identified and claimed as the invention.

We claim:

1. A dynamic respiratory disease diagnostic application, comprising in combination:
an input module to capture audio data including voice data and cough data and demographic data for a patient;
a transformation module for generating a waveform and a spectrogram from the captured audio data;
an Artificial Intelligence (AI) module for analyzing the generated spectrogram with a Convolutional Neural Network (CNN) module;
the Convolutional Neural Network (CNN) module for analyzing the spectrogram and extract disease-specific spectral features;
a waveform analysis module for calculating Zero Rate Crossing (ZCR), Chroma Features, Spectral Contrast, Magnitude, Root Mean Square (RMS), and Short-time Fourier Transform (STFT) as waveform features for additional cough biomarkers analysis or disease biomarkers analysis;
a classification module for combing the AI CNN extracted disease-specific spectral features and the calculated waveform features and for applying dense layers to classify one or more disease types based on one or more cough types and cough and disease biomarkers;
a temperature scaling module for calculating disease probabilities based on the one or more cough types and the one or more disease categories classified by the classification module; and
an output module for displaying output information on a graphical user interface (GUI) including a cough biomarker analysis, a disease or illness type based on the one or more cough types and the one or more disease categories classified by the classification module and a probability of the disease type or illness type calculated by the temperature scaling module.

2. The dynamic respiratory disease diagnostic application of claim 1, wherein the dynamic diagnosis respiratory disease diagnostic application includes a software application, firmware application, hardware application or a Software as a Service (SaaS) application for a cloud communications network.

3. The dynamic respiratory disease diagnostic application of claim 1, wherein the Artificial Intelligence (AI) module includes Generative AI methods, models and large language models (LLMs) for automatic Generative AI diagnosis of respiratory diseases.

4. The dynamic respiratory disease diagnostic application of claim 1, wherein the Artificial Intelligence (AI) module includes Predictive AI methods, models and large language models (LLMs) for automatic Predictive AI diagnosis of respiratory diseases.

5. A method for dynamic diagnosis of respiratory diseases, comprising:
capturing audio data from a patient on an input module on a server respiratory disease diagnosis application on a server network device with one or more processors or from a respiratory disease diagnosis application on a target network device with one or more processors via a communications network;
transforming on a transformation module on the server respiratory disease diagnosis application on the server network device, the captured the audio data into a waveform and spectrogram;
extracting on a waveform analysis module on the server respiratory disease diagnosis application on the server network device, spectral and temporal features from the waveform as one or more cough types or disease biomarkers;
analyzing with Artificial Intelligence (AI) application and a Convolutional Neural Network (CNN) module on the server respiratory disease diagnosis application on the server network device, the spectrogram to detect disease indicators;
classifying with a classification module on the server respiratory disease diagnosis application on the server network device, the captured audio data into one or more cough types and one or more disease categories based on extracted features;
calculating with a temperature scaling module on the server respiratory disease diagnosis application on the server network device, disease probabilities based on the one or more cough types and one or more disease categories classified by the classification module;
creating a final report on an output module on the server respiratory disease diagnosis application on the server network device, including the classified one or more cough types and one or more disease categories classified by the classification module and the calculated disease probabilities calculated by the temperature scaling module; and
displaying securely on the server respiratory disease diagnosis application on the server network device, the created final diagnostic report.

6. The method of claim 5, wherein the server respiratory disease diagnosis application and the respiratory disease diagnosis application comprise:
an input module to capture audio data including voice data and cough data and demographic data for a patient;
a transformation module for generating a waveform and a spectrogram from the captured audio data;
an Artificial Intelligence (AI) module for analyzing the generated spectrogram with a Convolutional Neural Network (CNN) module;
the Convolutional Neural Network (CNN) module for analyzing the spectrogram and extract disease-specific spectral features;
a waveform analysis module for calculating Zero Rate Crossing (ZCR), Chroma Features, Spectral Contrast, Magnitude, Root Mean Square (RMS), and Short-time Fourier Transform (STFT) as waveform features for additional cough biomarkers analysis or disease biomarkers analysis;
a classification module for combing the AI CNN extracted disease-specific spectral features and the calculated waveform features and for applying dense layers to classify one or more disease types based on one or more cough types and cough and disease biomarkers;
a temperature scaling module for calculating disease probabilities based on the one or more cough types and the one or more disease categories classified by the classification module; and
an output module for displaying output information on a graphical user interface (GUI) including a cough biomarker analysis, a disease or illness type based on the one or more cough types and the one or more disease categories classified by the classification module and a probability of the disease type or illness type calculated by the temperature scaling module.

7. The method of claim 5, further comprising:
displaying securely the created final diagnostic report on the respiratory disease diagnosis application on the target network device via the communications network.

8. The method of claim 5, further comprising:
sending an electronic link for the created final diagnostic report stored on the server network device from the server respiratory disease diagnosis application on the server network device to the respiratory disease diagnosis application on the target network device via the communications network.

9. The method of claim 5, further comprising:
sending the created final diagnostic report from the server respiratory disease diagnosis application on the server network device to the respiratory disease diagnosis application on the target network device with via the communications network with one or more secure messages; and
sending the created final diagnostic report from the server respiratory disease diagnosis application on the server network device to an online patient portal, an online medical facility medical record system or an online medical record billing system via the communications network with one or more secure messages.

10. The method of claim 9 wherein, the sending steps include sending one or more secure messages including: an email message, voice message, video message, RCS message, Short Message Service (SMS) message, Direct Message (DM), Instant Message (IM), Multimedia Messaging Service (MMS) message, GOOGLE Business Message, APPLE iMessage, instant message, direct message, Short Message Peer-to-Peer (SMPP) message, social media message, REpresentational State Transfer (REST) message, data link protocol message, network protocol message, Simple Object Access Protocol (SOAP) message, or Lightweight Directory Access Protocol (LDAP) message, using one or more encryption or security methods.

11. The method of claim 9 wherein, the one or more secure messages are securely sent and securely received and the with one or more of: a Wireless Encryption Protocol (WEP), Advanced Encryption Standard (AES), Data Encryption Standard (DES), RSA encryption, Secure Hash Algorithm (SHA), Message Digest-5 (MD-5), Keyed Hashing for Message Authentication Codes (HMAC), Electronic Code Book (ECB) or Diffie and Hellman (DH) or Secure Sockets Layer (SSL), encryption or security methods.

12. The method of claim 9, wherein all secure message communications between the server respiratory disease diagnosis application on the server network device to the respiratory disease diagnosis application on the target network device via the communications network include secure end-to-end encryption.

13. The method of claim 5, further comprising:
creating on the server respiratory disease diagnosis application on the server network device one or more medical diagnostic codes for the one or more cough types and one or more disease categories included in the final diagnostic report.

14. The method of claim 5, further comprising:
creating on the server respiratory disease diagnosis application on the server network device one or more medical billing codes for medical procedures performed on the patient or medical services provided to the patient based the one or more cough types and one or more disease categories included in the final diagnostic report.

15. The method of claim 5, further comprising:
providing with the server respiratory disease diagnosis application on the server network device provides telehealth remote patients via the communications network; and
providing providing with the server respiratory disease diagnosis application on the server network device provides telehealth remote patients via the communications network.

16. The method of claim 5 wherein, target network device and the server network device include one or more wireless communications interfaces comprising one or more of: a cellular telephone, 802.11a, 802.11b, 802.11g, 802.11n, 802.11ac, 802.11ax, 802.11be, 802.15.4 (ZigBee), Wireless Fidelity (Wi-Fi), Wi-Fi Aware, Worldwide Interoperability for Microwave Access (WiMAX), ETSI High Performance Radio Metropolitan Area Network (HIPERMAN), Near Field Communications (NFC), Machine-to-Machine (M2M), 802.15.1 (BLUETOOTH®), RFID, or infra data association (IrDA), wireless or communication interfaces.

17. The method of claim 5 wherein, the target network device includes: desktop and laptop computers, tablet computers, mobile phones, non-mobile phones with displays, smart phones, Internet phones, Internet appliances, personal digital/data assistants (PDA), portable, handheld and desktop video game devices, cable television (CATV), satellite television (SATV) and Internet television set-top boxes, digital televisions including high definition television (HDTV), three-dimensional (3DTV) televisions, smart speakers, Internet of Things (IoT) devices, Radio Frequency Identifier (RFID) devices, or wearable network devices, with wireless or wired network interfaces, connectable to the communications network.

18. One or more non-transitory computer readable mediums each having stored therein a plurality of instructions for causing one or more processors on one more network devices to execute the steps of:
an input module to capture audio data including voice data and cough data and demographic data for a patient;
a transformation module for generating a waveform and a spectrogram from the captured audio data;
an Artificial Intelligence (AI) module for analyzing the generated spectrogram with a Convolutional Neural Network (CNN) module;
the Convolutional Neural Network (CNN) module for analyzing the spectrogram and extract disease-specific spectral features;
a waveform analysis module for calculating Zero Rate Crossing (ZCR), Chroma Features, Spectral Contrast, Magnitude, Root Mean Square (RMS), and Short-time Fourier Transform (STFT) as waveform features for additional cough biomarkers analysis or disease biomarkers analysis;
a classification module for combing the AI CNN extracted disease-specific spectral features and the calculated waveform features and for applying dense layers to classify one or more disease types based on one or more cough types and cough and disease biomarkers;
a temperature scaling module for calculating disease probabilities based on the one or more cough types and the one or more disease categories classified by the classification module; and
an output module for displaying output information on a graphical user interface (GUI) including a cough biomarker analysis, a disease or illness type based on the one or more cough types and the one or more disease categories classified by the classification module and a probability of the disease type or illness type calculated by the temperature scaling module; and
displaying securely the created final diagnostic report on the respiratory disease diagnosis application on the target network device via the communications network.

19. A system for dynamic diagnosis of respiratory diseases, comprising in combination:
one or more target network devices, each with one or more processors,
one or more server network devices, each with one or more processors;
a communications network;
for capturing audio data from a patient on an input module on a server respiratory disease diagnosis application on a server network device with one or more processors or from a respiratory disease diagnosis application on a target network device with one or more processors via a communications network;
for transforming on a transformation module on the server respiratory disease diagnosis application on the server network device, the captured the audio data into a waveform and spectrogram;
for extracting on a waveform analysis module on the server respiratory disease diagnosis application on the server network device, spectral and temporal features from the waveform as one or more cough types or disease biomarkers;
for analyzing with Artificial Intelligence (AI) application and a Convolutional Neural Network (CNN) module on the server respiratory disease diagnosis application on the server network device, the spectrogram to detect disease indicators;
for classifying with a classification module on the server respiratory disease diagnosis application on the server network device, the captured audio data into one or more cough types and one or more disease categories based on extracted features;
for calculating with a temperature scaling module on the server respiratory disease diagnosis application on the server network device, disease probabilities based on the one or more cough types and one or more disease categories classified by the classification module;
for creating a final report on an output module on the server respiratory disease diagnosis application on the server network device, including the classified one or more cough types and one or more disease categories classified by the classification module and the calculated disease probabilities calculated by the temperature scaling module;
for displaying securely on the server respiratory disease diagnosis application on the server network device, the created final diagnostic report; and
for displaying securely on the respiratory disease diagnosis application on the target network device the created final diagnostic report, via the communications network.

20. The system of claim 19, wherein the server respiratory disease diagnosis application and the respiratory disease diagnosis application comprise:
an input module to capture audio data including voice data and cough data and demographic data for a patient;
a transformation module for generating a waveform and a spectrogram from the captured audio data;
an Artificial Intelligence (AI) module for analyzing the generated spectrogram with a Convolutional Neural Network (CNN) module;
the Convolutional Neural Network (CNN) module for analyzing the spectrogram and extract disease-specific spectral features;
a waveform analysis module for calculating Zero Rate Crossing (ZCR), Chroma Features, Spectral Contrast, Magnitude, Root Mean Square (RMS), and Short-time Fourier Transform (STFT) as waveform features for additional cough biomarkers analysis or disease biomarkers analysis;
a classification module for combing the AI CNN extracted disease-specific spectral features and the calculated waveform features and for applying dense layers to classify one or more disease types based on one or more cough types and cough and disease biomarkers;
a temperature scaling module for calculating disease probabilities based on the one or more cough types and the one or more disease categories classified by the classification module; and
an output module for displaying output information on a graphical user interface (GUI) including a cough biomarker analysis, a disease or illness type based on the one or more cough types and the one or more disease categories classified by the classification module and a probability of the disease type or illness type calculated by the temperature scaling module.
